(19) United States
(12) Patent Application Publication
(10) Pub. No.: US 2003/0096432 A1
(43) Pub. Date: May 22, 2003

(54) Screening methods

(75) Inventor: Bent Karsten Jakobsen, Wantage (GB)

Correspondence Address: HALE AND DORR, LLP, 60 STATE STREET, BOSTON, MA, 02109

(73) Assignee: AVIDEX LIMITED, Milton (GB)

(21) Appl. No.: 10/103,597

(22) Filed: Mar. 21, 2002

Related U.S. Application Data
(63) Continuation of application No. PCT/GB00/03579, filed on Sep. 18, 2000.

(30) Foreign Application Priority Data
Sep. 21, 1999 (GB) 9922352.1

(51) Int. Cl. G01N 33/543

(52) U.S. Cl. 436/518

(57) ABSTRACT

The present invention provides methods for sequentially screening for compounds with the potential to interfere with low affinity receptor-ligand contacts using an interfacial optical assay, such as surface plasmon resonance (SPR). The method comprises contacting a candidate compound with an immobilised receptor, contacting the receptor, which may or may not have the candidate compound bound to it, with the ligand and detecting by interfacial optical assay whether or not the ligand or ligand-compound complex has bound to the receptor or receptor-compound complex. If the ligand binds, the method shows that the compound does not inhibit the receptor-ligand interaction. If the ligand does not bind, the method shows that the compound inhibits the receptor-ligand interaction. The method is particularly usefull for screening for inhibitors of the interaction between MHC/peptide complex and T cell receptor, MHC/peptide complex and CD8 coreceptor or MHC/peptide complex and CD4 coreceptor.

The present invention relates to methods of screening and, in particular, to methods of screening libraries of candidate compounds for those which inhibit the binding of a low, affinity receptor-ligand interaction having fast binding kinetics.

A vast number of cellular interactions and cell responses are controlled by contacts made between cell surface receptors and soluble ligands, or ligands presented on the surfaces of other cells. These types of specific molecular contacts are of crucial importance to the correct biochemical regulation in the human body and are therefore being studied intensely. In many cases, the objective of such studies is to devise a means of modulating cellular responses in order to prevent or combat disease.

In this regard, chemical or biochemical compounds with the ability to bind specifically to a particular cell surface molecule, or to a soluble ligand which is recognised by a cell surface receptor, can have potential for a multitude of therapeutic purposes. For instance, a compound with specificity for a certain ligand may inhibit or prevent a cellular response transduced through the corresponding cell surface receptor. Therefore, methods with which to identify compounds that bind with some degree of specificity to human receptor or ligand molecules are important as leads for the discovery and development of new disease therapeutics. In particular, compounds that interfere with certain receptor-ligand interactions have immediate potential as therapeutic agents or carriers.

Most attention is focussed on the identification of small, that is, low molecular weight, compounds with therapeutic potential. This is generally because such compounds: are usually inexpensive to produce; can often be relatively easily and swiftly modified so as to provide variants of a “lead compound” which may have different properties; are often relatively stable, or can be modified to be stable, in the body, in particular compared to proteins and other biochemical substances; are less likely to provoke unwanted physiological reactions, like immune responses, than larger entities; and are more likely to be able to be administered orally because they are more likely to be able to pass the membrane barriers of the digestive tract into the blood, while less likely to be degraded by the digestive system.

Recent advances in combinatorial chemistry, enabling relatively easy and cost-efficient production of very large compound libraries, has increased the scope for compound testing enormously. Now the limitations of screening programmes most often reside in the nature of the assays that can be employed and, in particular, how well these assays can be adapted to high throughput screening methods.

Many cell surface receptor-ligand contacts are characterised by low affinity interactions and fast binding kinetics (Van der Merwe et al J. Exp. Med. 185:393-403 (1997)). Binding affinity is related to the speed of the binding kinetics, i.e. it is a function of the off rate compared to the on rate. Thus, it is theoretically possible to Ahave a low affinity interaction in which the off rate is very low but the on rate is even lower (and conversely a high affinity interaction in which the off rate is very high but the on rate is even higher). However, interactions with fast binding kinetics generally have a relatively high on rate and an even higher off rate. The off rate may be in the range of from 0.001 s⁻¹ to 1000 s⁻¹, preferably about 0.01 s⁻¹ to 100 s⁻¹. For example, T cell receptors (TCR) have an off rate of approximately 0.05 s⁻¹ from an MHC/peptide complex and CD8 has an off rate of approximately 10 s⁻¹ from an MHC/peptide complex. Such interactions may have a Kd in the range of 0.1 μM or less to 10 mM or more, and possibly about 1 μM to 1 mM. For example, the interaction between a TCR and an MHC/peptide complex is of the order of 10 μM, while that between CD8 and an MHC/peptide complex is of the order of 0.5 mM. Interactions having K_{d}s above 10 mM tend to be non-specific. Because low affinity interactions having fast binding kinetics are so brief and weak, they are very difficult to detect.

The scintillation proximity assay (SPA) has been used to screen compound libraries for inhibitors of the low affinity interaction between CD28 and B7 (K_{d} probably in the region of 4 μM (Van der Merwe et al J. Exp. Med. 185:393-403 (1997), Jenh et al, Anal Biochem 165(2) 287-93 (1998)). SPA is a radioactive assay making use of beta particle emission from certain radioactive isotopes which transfers energy to a scintillant immobilised on the indicator surface. The short range of the beta particles in solution ensures that scintillation only occurs when the beta particles are emitted in close proximity to the scintillant. When applied for the detection of protein-protein interactions, one interaction partner is labelled with the radioisotope, while the other is either bound to beads containing scintillant or coated on a surface together with scintillant. If the assay can be set up optimally, the radioisotope will be brought close enough to the scintillant for photon emission to be activated only when binding between the two proteins occurs.

However, SPA suffers from a number of problems which limits its general use for high throughput screening for inhibitors of receptor-ligand interactions. Indeed, there are very few reports of the use of SPA for screening. The assay requires radioactive labelling of one of the interaction partners, a modification which may not be achievable without affecting its binding specificity towards its interaction partner. There are also many technical difficulties involved in developing reliable SPA protocols for many receptor-ligand interactions. The nature of SPA makes it sensitive to even small variations in the reaction conditions in the individual wells used for compound library screening. Particularly where protein-protein interactions which are characterised by fast kinetics are concerned, the assay is vulnerable to experimental variation. Where such proteins are involved, a relatively low proportion of the scintillant will be activated due to the transient nature of the protein-protein contacts, and thus variations in the assay can easily cause the readout to vary unacceptably. A further drawback of SPA is that it relies on the use of dangerous substances, i.e. radioisotopes and scintillation liquid which have to be disposed of safely.

The present inventors have devised a strategy for screening for compounds with the potential to interfere with low affinity receptor-ligand contacts using an interfacial optical assay, such as surface plasmon resonance (SPR).

According to the present invention, there is provided a method of sequentially screening candidate compounds for compounds with the ability to inhibit a receptor-ligand interaction having fast binding kinetics, the method comprising the steps of:

a) optionally contacting the receptor with the ligand, the receptor being immobilised so that binding of the ligand therewith can be detected in an interfacial optical assay, detecting by interfacial optical assay the binding of the ligand to the receptor, and washing the ligand from the receptor;

b) contacting an n^{th} candidate compound with the ininobilised receptor;

c) optionally washing the receptor at a predetermined stringency to remove the n^{th} candidate compound if it has too low an affinity for the receptor;

d) contacting the receptor, which may or may not have the n^{th} candidate compound bound to it, with the ligand, and detecting by interfacial optical assay whether or not the ligand or ligand-compound complex has bound to the receptor or receptor-compound complex; and

e) either i) if the ligand has bound, deducing that the n^{th} compound does not inhibit the receptor-ligand interaction, optionally washing the receptor, incrementing n, and returning to optional step a) or step b), or ii) if the ligand has not bound, deducing that the n^{th} compound inhibits the receptor-ligand interaction.

The present invention and preferred embodiments thereof will now be described in more detail. Reference is made to the accompanying drawings in which:

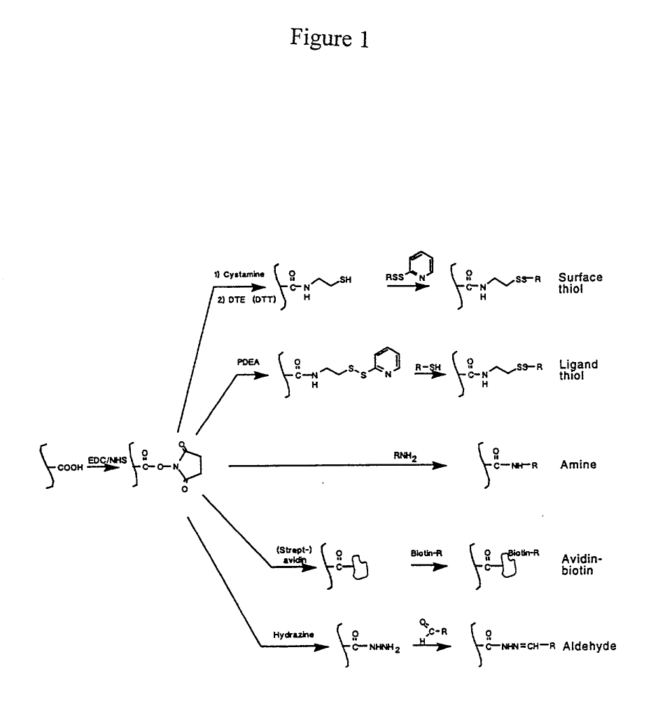

FIG. 1 is a diagram summarising methods by which soluble proteins can be immobilised on the surface of BIAcore surface plasmon resonance chips;

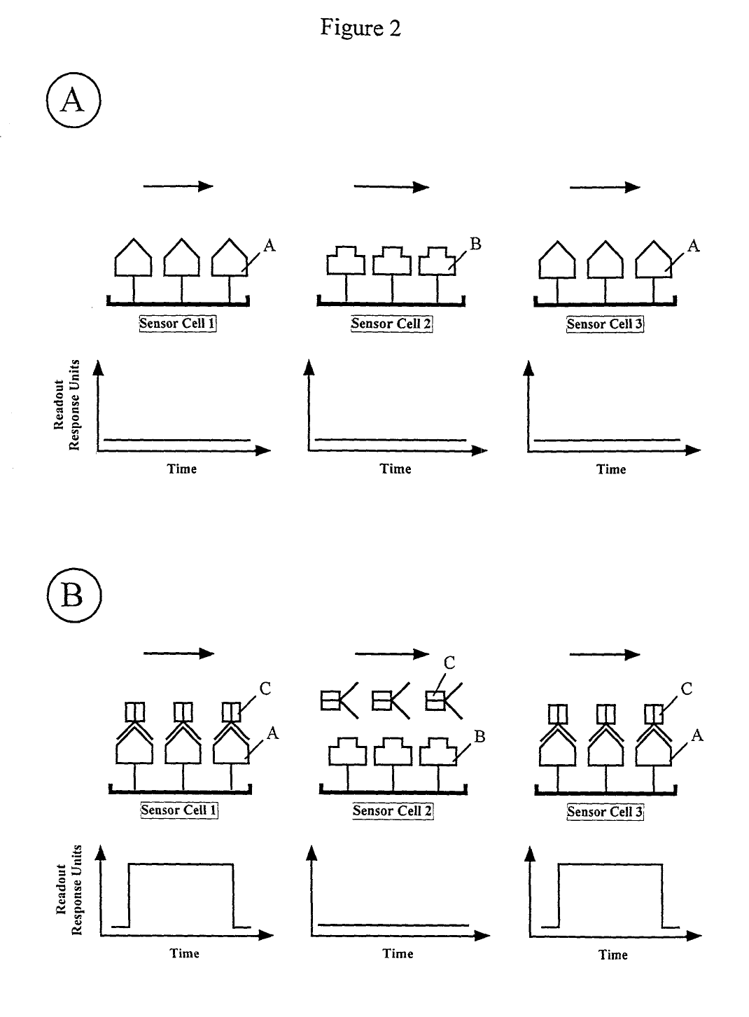

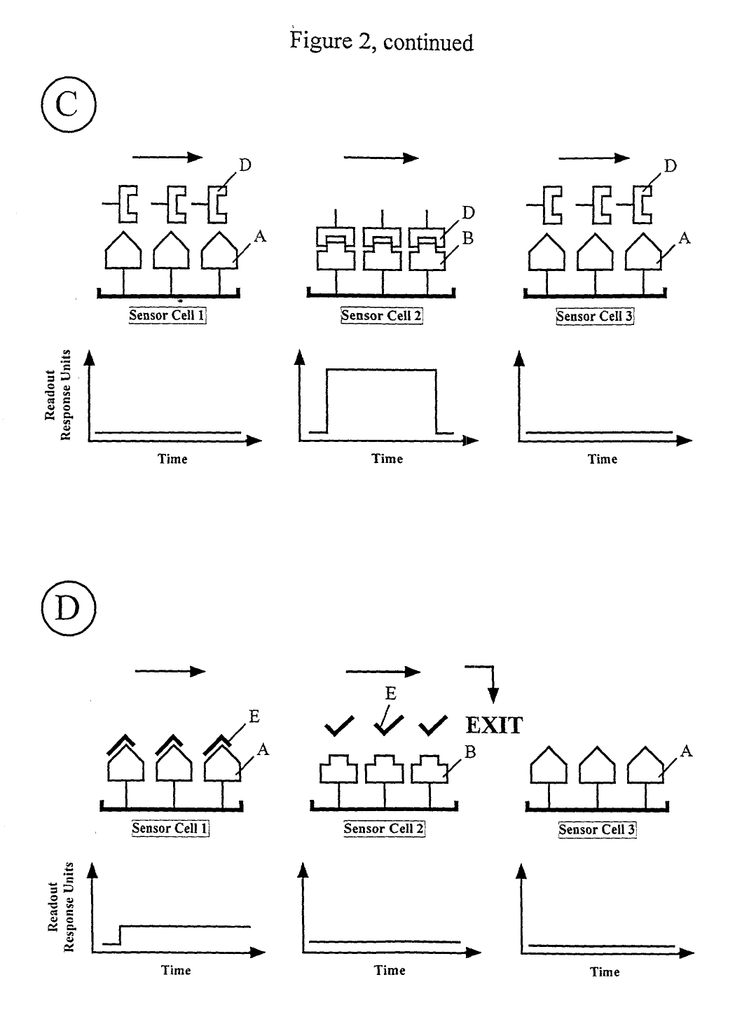

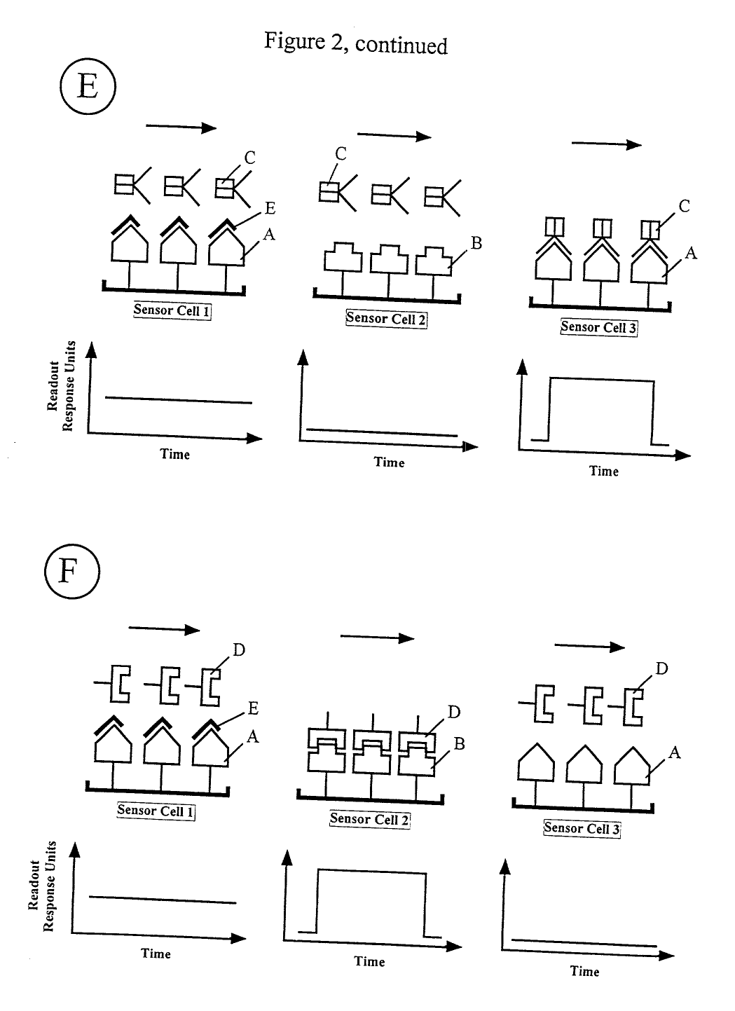

FIG. 2 is a schematic representation of the steps in one method for screening in accordance with the present invention, using SPR;

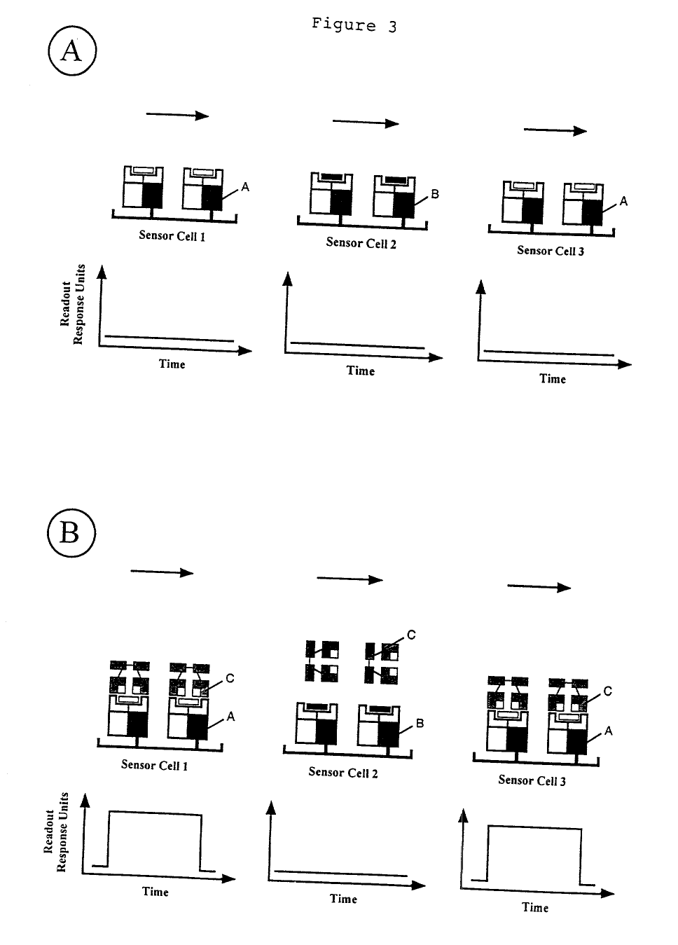

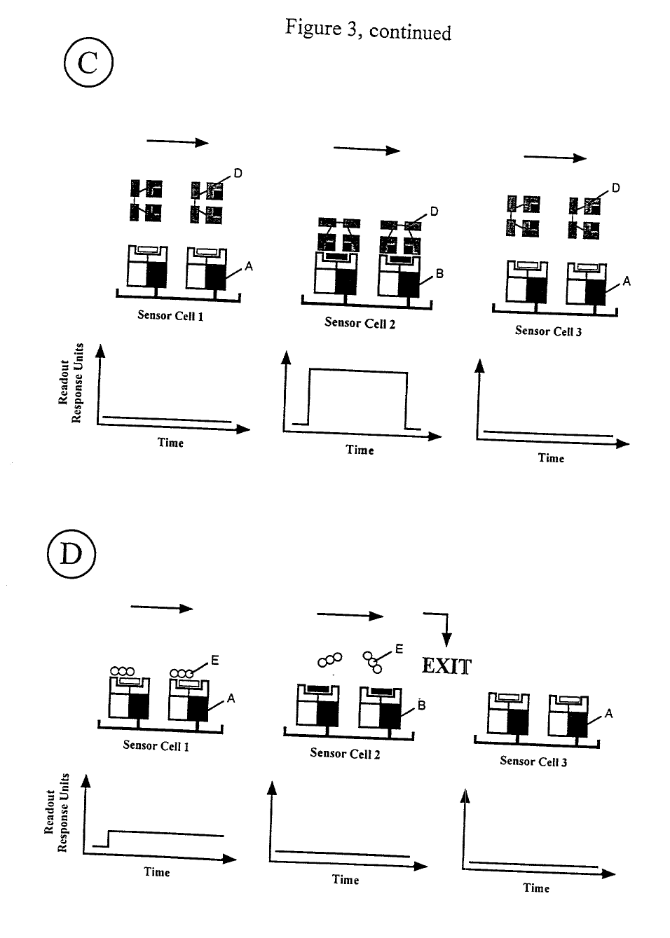

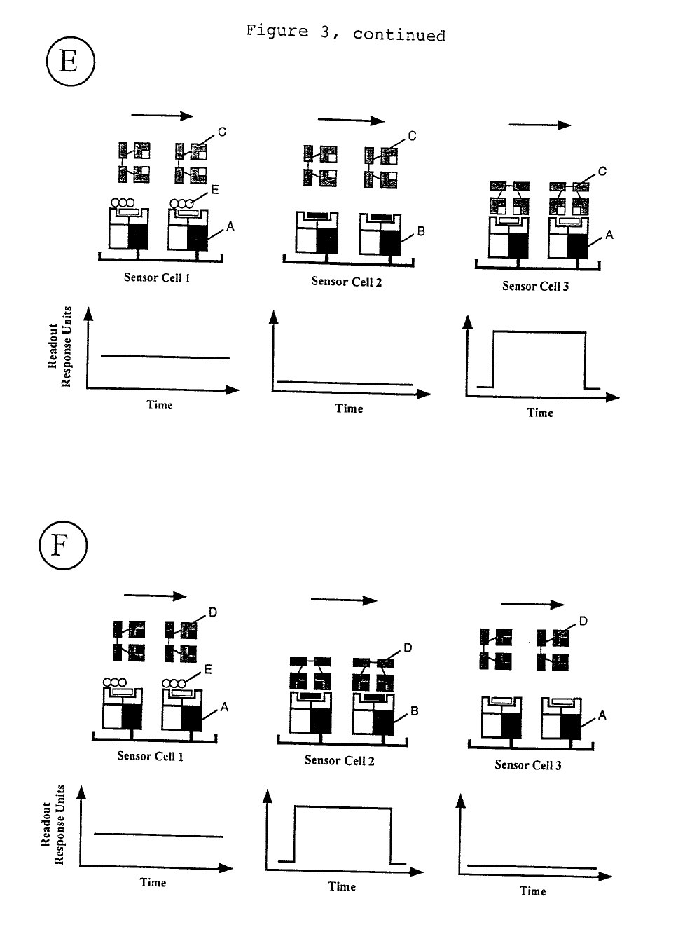

FIG. 3 is a schematic representation of the steps in a method in accordance with the present invention for screening for an inhibitor which inhibits the binding of a T cell receptor to an MHC molecule complexed with a specific peptide antigen;

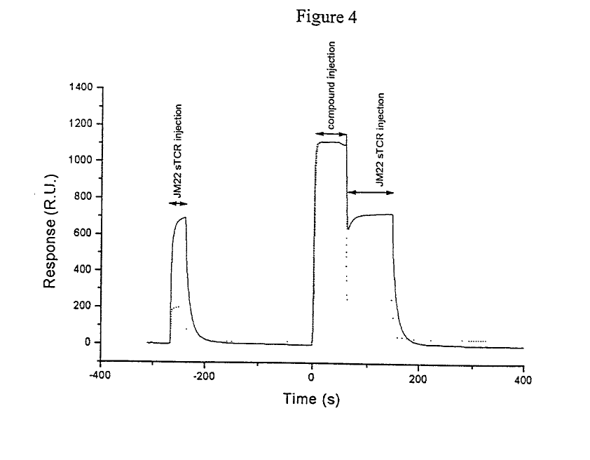

FIG. 4 is a graph showing the response over time from binding of JM22 soluble TCR to flu-HLA-A2;

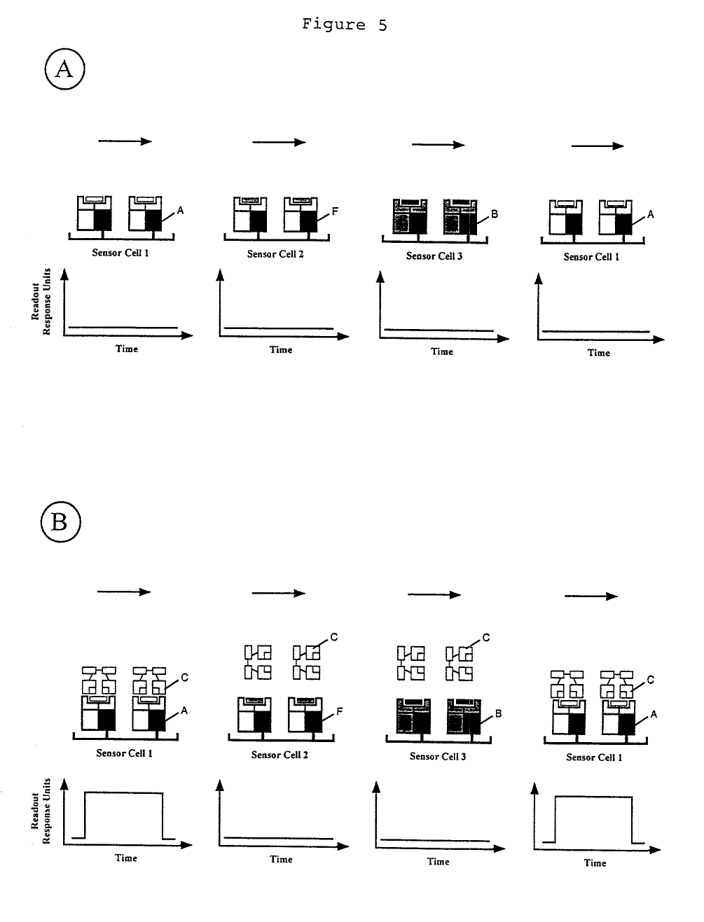

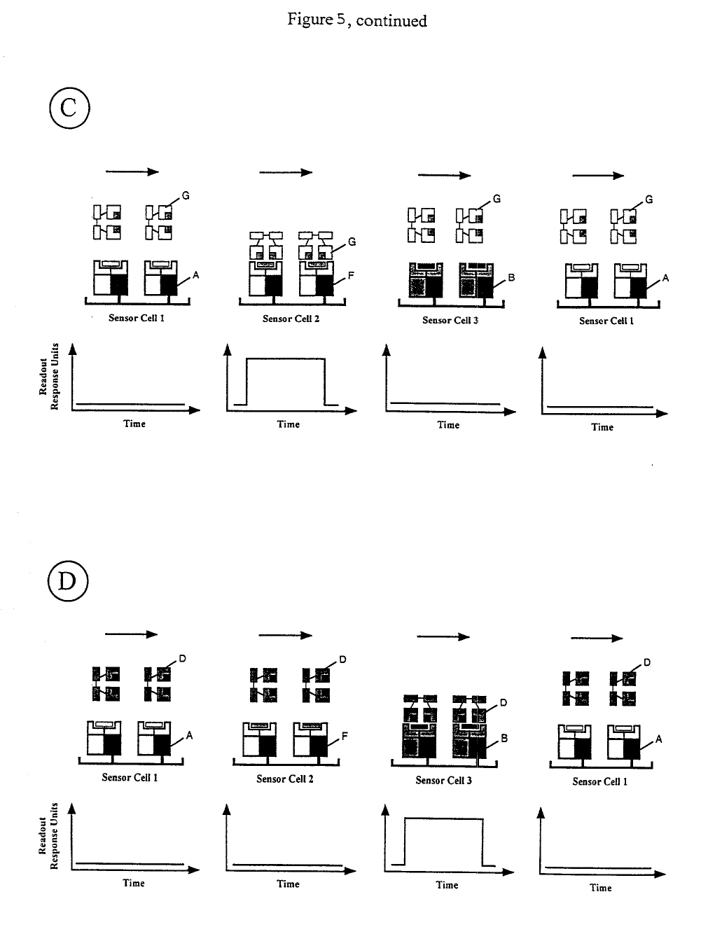

FIG. 5 is a schematic representation of the steps in one method in accordance with the present invention for screening for an inhibitor which inhibits the binding of T cell receptors to a particular MHC molecule, regardless of the antigen presented by that molecule;

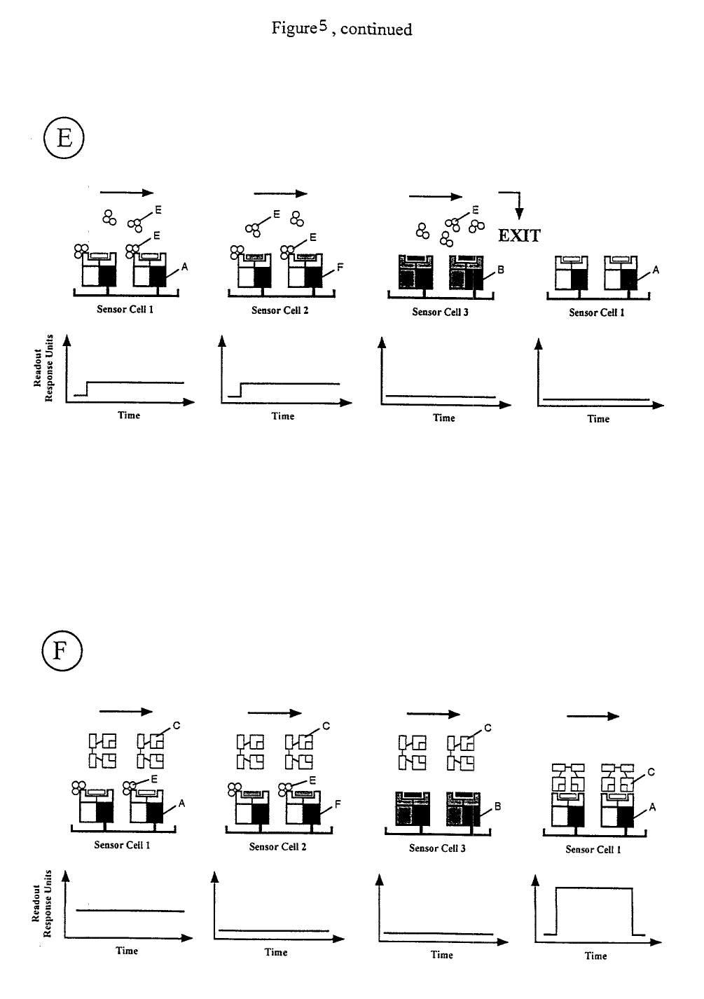

FIG. 6 is a schematic representation of the steps in one method in accordance with the present invention for screening for an inhibitor which inhibits the binding of CD8 to class I HLA molecules and an inhibitor which inhibits the binding of CD4 to class II HLA molecules;

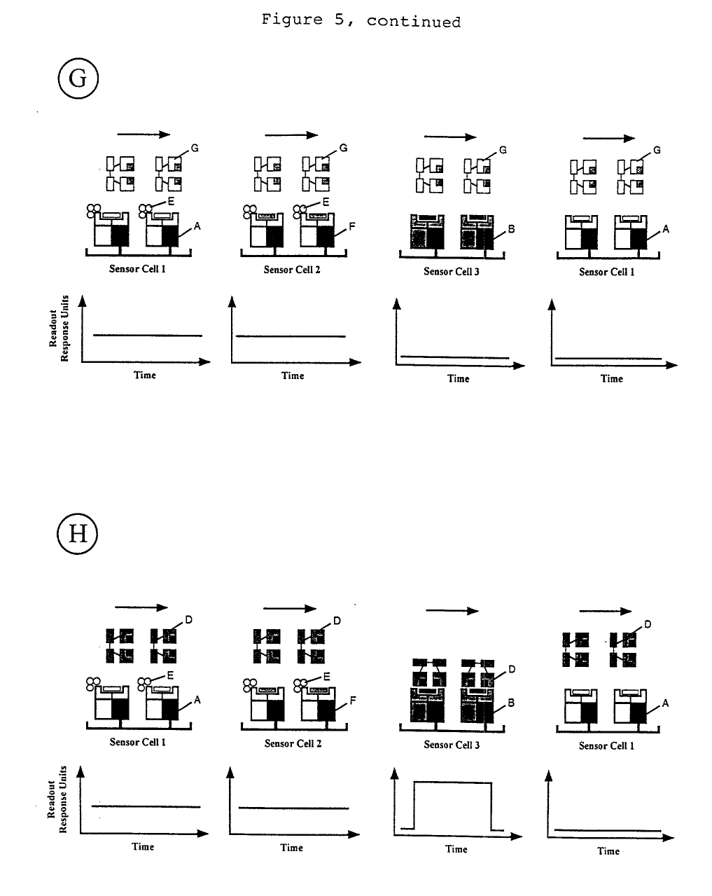

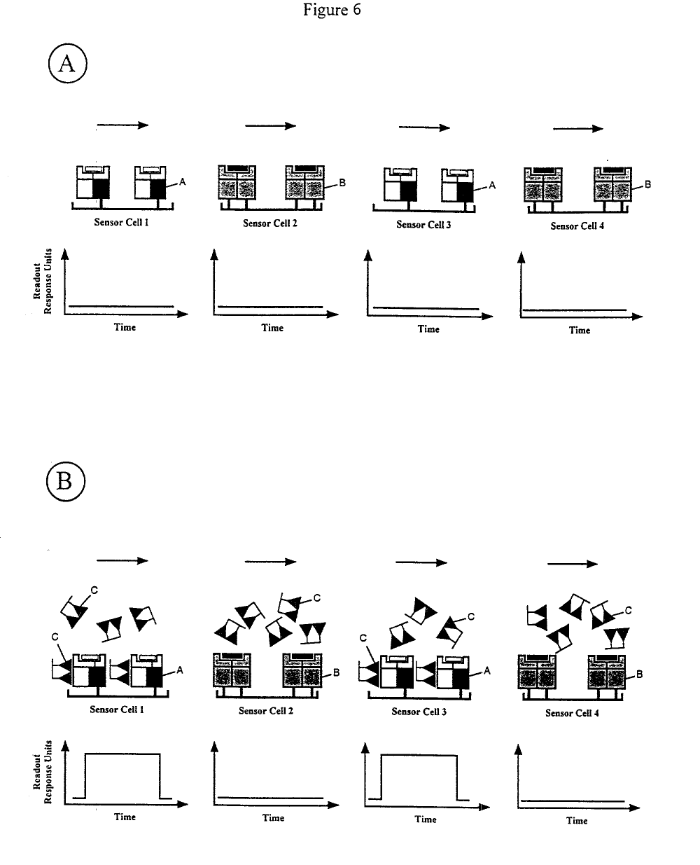

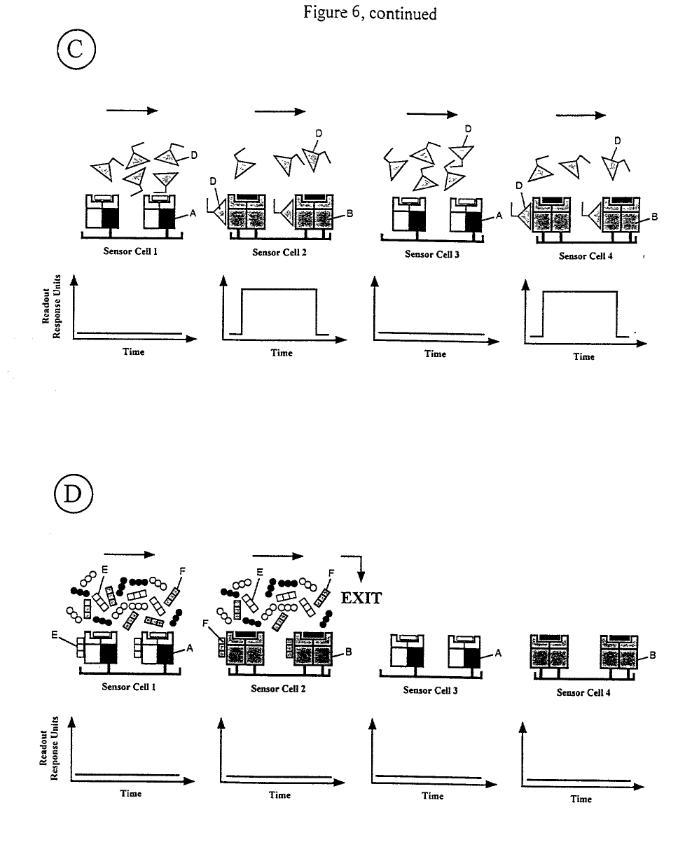

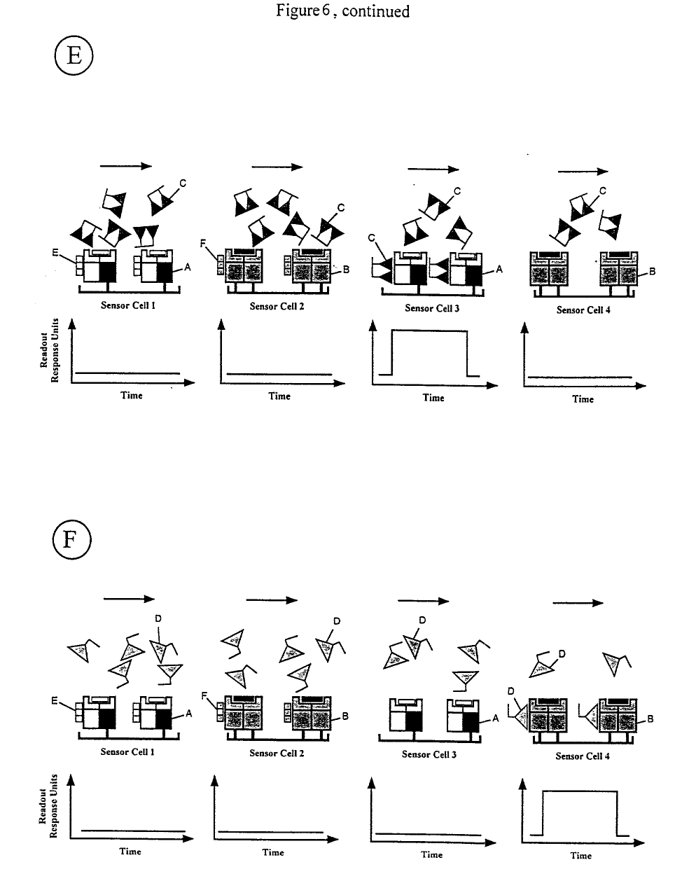

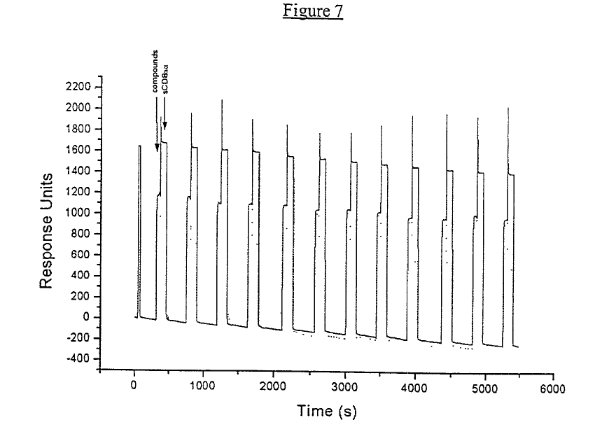

FIG. 7 is a BIAcore trace showing the response over time from binding of sCD8αα to HLA-A2 in the presence of 96 compounds;

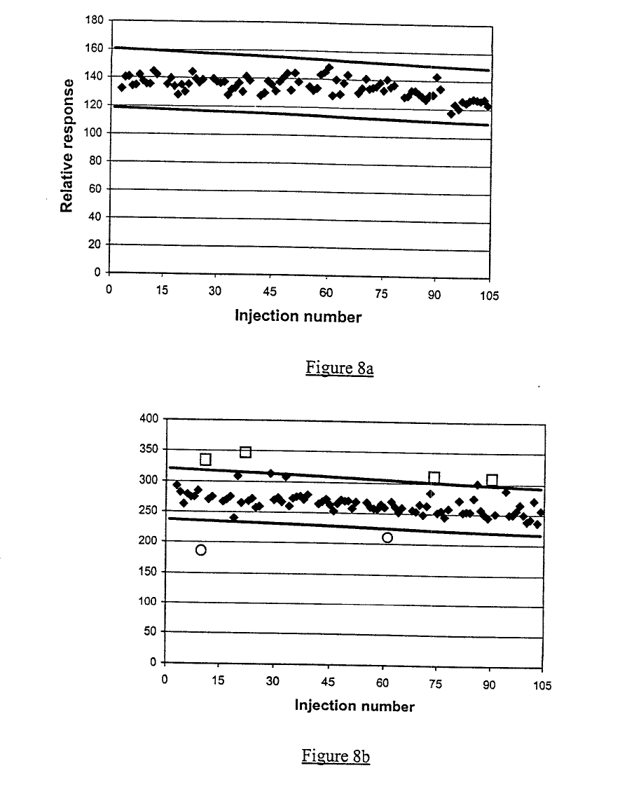

FIGS. 8a and 8b show the results of a BIAcore screen of potential inhibitors of the interaction between HLA-A2 and sCD8αα;

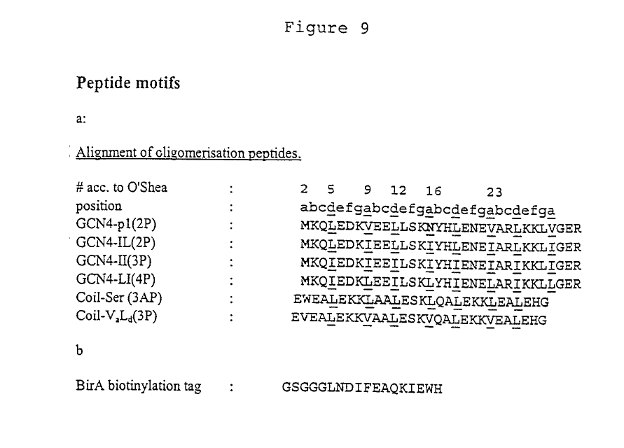

FIG. 9 shows the amino acid sequences of (a) leucine zippers and (b) of a BirA biotinylation tag (Schatz, Biotechnology N Y 11(10):1138-43 (1993));

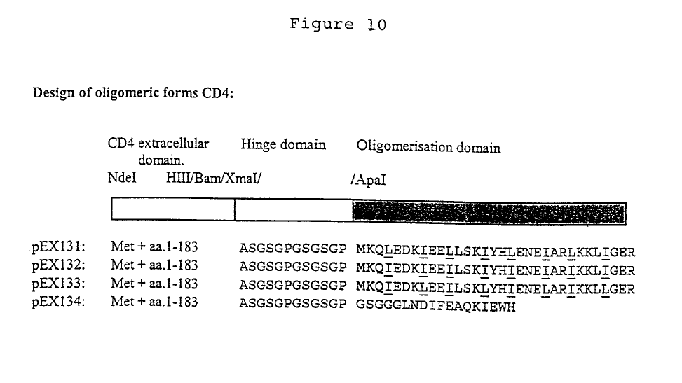

FIG. 10 illustrates alternative designs for CD4 oligomerisation fusion proteins;

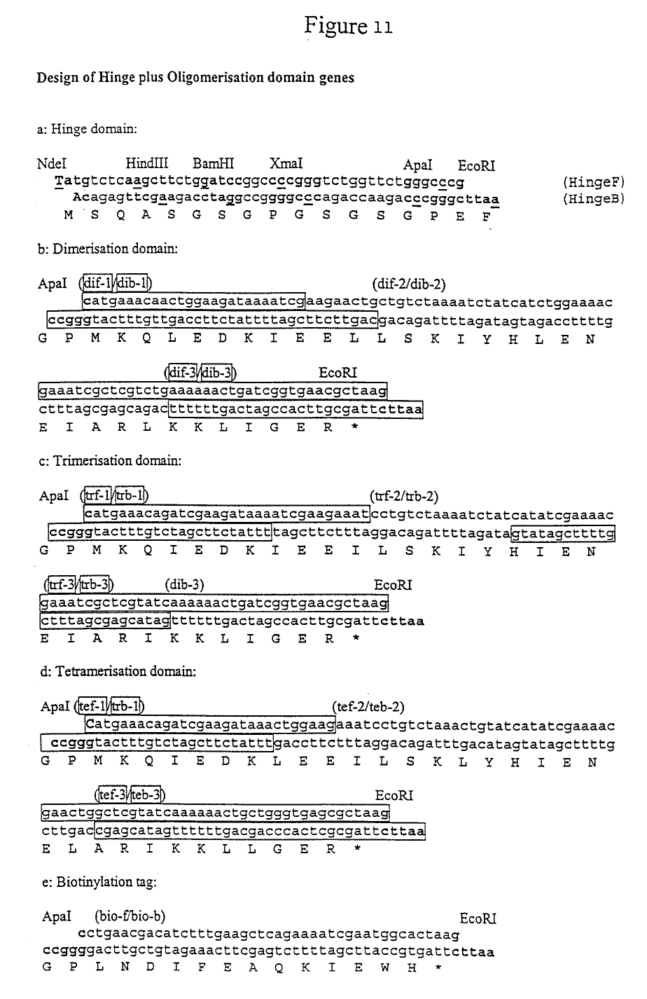

FIGS. 11a-e illustrate the nucleotide and amino acid sequences of the hinge and oligomerisation domains used for the construction of multimeric CD4;

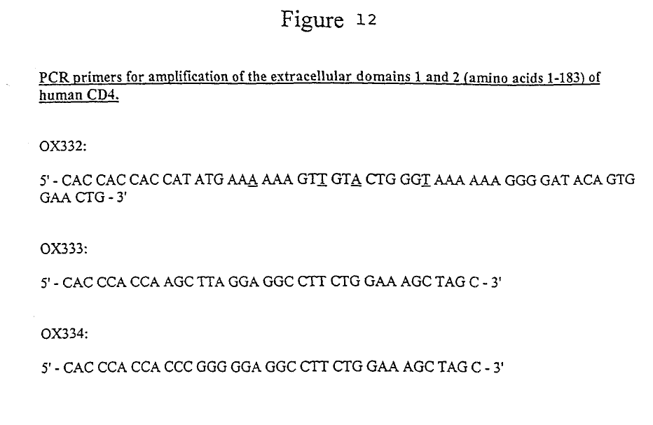

FIG. 12 shows the sequences of the primers used for amplification of the gene encoding the extracellular domains 1 and 2 of human CD4. The underlined nucleotides indicate silent mutations introduced in the 5′-end of the gene to facilitate expression initiation in E. coli; and

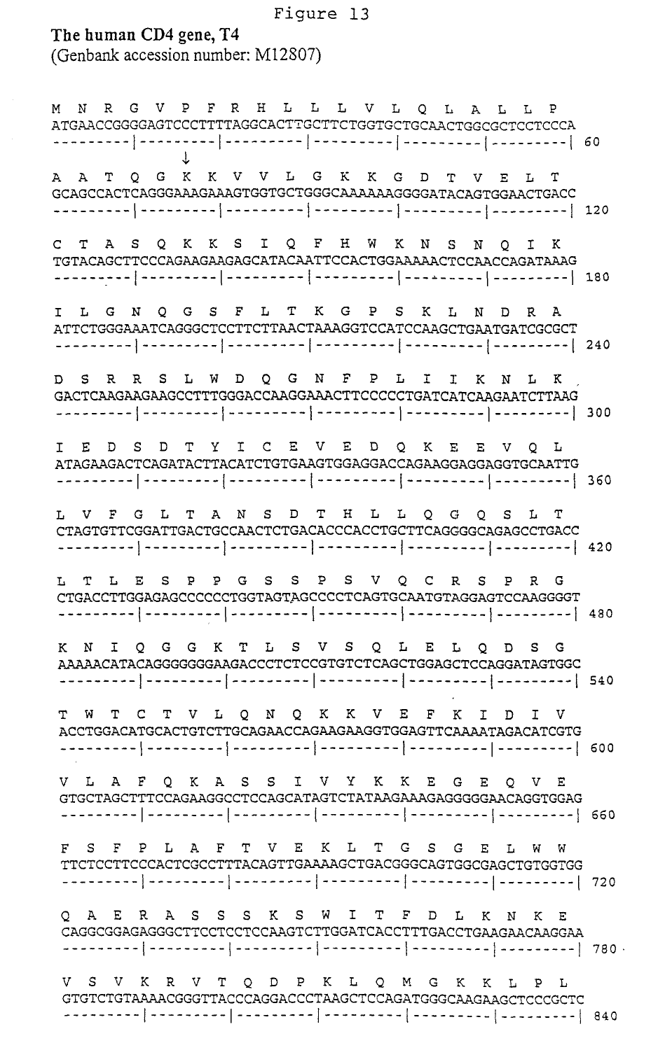

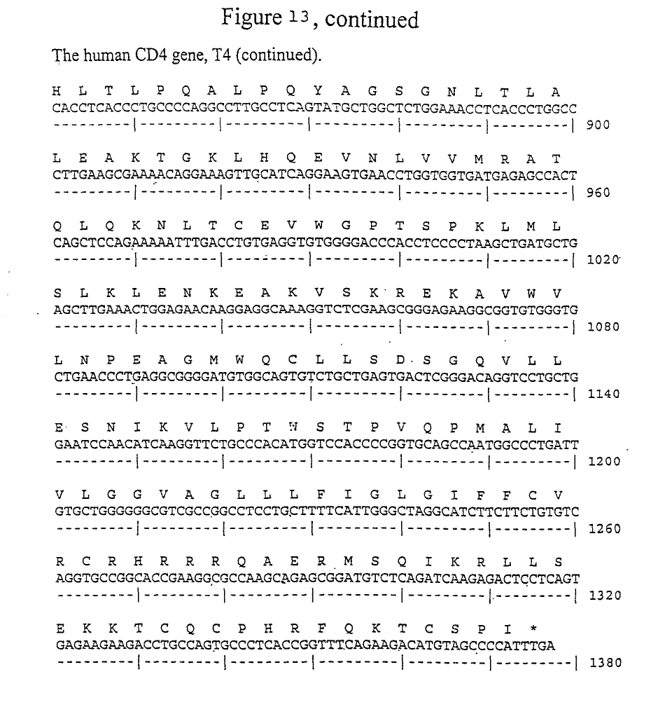

FIG. 13 shows the cDNA and protein sequence of the human CD4. The initial 25 amino acids constitute the signal peptide which is cleaved off during processing. The arrow indicates position +1 in the mature polypeptide.

Unless the context dictates otherwise, the terms “receptor” and “ligand” as used herein are intended to mean either one of two binding partners, the “ligand” being in soluble form and the “receptor” being immobilised for the interfacial optical assay and transducing a change in optical characteristics when the ligand binds thereto. It will therefore be appreciated that the term “receptor” as used herein may include what is conventionally referred to as a ligand, and the term “ligand” as used herein may include what is conventionally referred to as a receptor (where, for example, a receptor is a molecule which transduces a signal when the ligand binds to the receptor). The “receptor” and “ligand” may be proteins or other entities.

In the method of the present invention, an inhibitory compound is detected by monitoring whether the ligand binds to the receptor after exposure to the compound, rather than by monitoring binding of the compound to the receptor, which is difficult to detect in interfacial optical assays. This is because the change in refractive index detected in such assays is dependent on the change in mass. Thus, the binding of a small molecule to the receptor may not make a sufficient change to the mass to give a clear signal over the inherent noise in the system. The method of the present invention avoids the problem of determining the difference between the receptor with nothing bound to it and the receptor with merely the compound bound to it. There will be a greater difference, and in practice often a much greater difference, between the mass of the receptor with the compound bound to it and the mass of the receptor with the ligand bound to it.

Moreover, the method of the present invention—in which a single step is required to identify compounds which bind to a receptor and inhibit the binding of a ligand—avoids an additional step which is required in assays where only binding of the candidate compound to the receptor is detected. This additional step is to screen complexes between the receptor and those compounds that have been shown to bind to the receptor for their ability to bind to the ligand; compounds with the desired modulating activity would be selected for further analysis or development. Typically this takes the form of in vivo assays which are time-consuming and expensive. Even where the additional step does not require in vivo assays, for low affinity interactions the second step is difficult in practice because of the difficulty in detecting such interactions. The present invention simplifies the task by enabling both of these steps to be achieved in a single screen.

The fast binding kinetics nature of the interaction between the ligand and the receptor is such that binding is short-lived. The interaction may also have a low affinity. Thus, using an interfacial optical assay means that detection of receptor-ligand binding can be carried out quickly and detected in real time, allowing such comparisons to be sequential. This provides a number of advantages, which are discussed in more detail below.

“Interfacial optical assays” include surface plasmon resonance (SPR). In this technique, one binding partner (normally the receptor) is inimobilised on a ‘chip’ (the sensor surface) and the binding of the other binding partner (normally the ligand), which is soluble and is caused to flow over the chip, is detected. The binding of the ligand results in an increase in concentration of protein near to the chip surface which causes a change in the refractive index in that region. The surface of the chip is comprised such that the change in refractive index may be detected by surface plasmon resonance, an optical phenomenon whereby light at a certain angle of incidence on a thin metal film produces a reflected beam of reduced intensity due to the resonant excitation of waves of oscillating surface charge density (surface plasmons). The resonance is very sensitive to changes in the refractive index on the far side of the metal film, and it is this signal which is used to detect binding between the immobilised and soluble proteins. Systems which allow convenient use of SPR detection of molecular interactions, and data analysis, are commercially available. Examples are the Iasys machines (Fisons) and the Biacore machines. The Biacore 2000™ system, for example, utilises a sensor chip consisting of four 0.02 μl flow cells. Each of these contains an optical surface to which is attached a very thin gold film to induce SPR, and a dextran matrix to which biomolecules can be immobilised. The reactant of interest is caused to flow over the chip surface and the binding to the immobilised biomolecules is detected by the mass increase proximal to the surface which leads to a change in the refractive index in that region.

Other interfacial optical assays include total internal reflectance fluorescence (TIRF), resonant mirror (RM) and optical grating coupler sensor (GCS), and are discussed in more detail in Woodbury and Venton (J. Chromatog. B. 725 113-137 (1999)).

Woodbury and Venton also discuss the applications of interfacial optical assays including SPR, and refer to papers in which SPR has been used to detect certain interactions. For example, Cheskis & Freedman (Biochem. 35(10):3309-18. (1996)) report the examination of DNA-protein interactions and their small-molecule modulators using SPR. In this report, the affinity of the interaction measured was relatively high (approximately 0.2-5 nM). Although Woodbury and Venton suggest that the work of Cheskis and Freedman could be adapted for high through-put screening, this is not credible because several hours would be required for the ligand to clear from the sensor chip before a further round of screening could be performed.

Woodbury and Venton also describe the work of Wiekowski et al. (Eur J Biochem 246(3):625-32 (1997)). Here, two small molecule inhibitors of the binding of interleukins to their receptors were identified. However, an interfacial optical assay was not used for this purpose; rather SPA was used. The inhibition of this binding then was checked using SPR, which, as noted above, is an interfacial optical assay. Woodbury and Venton consider this to be “one of the few literature reports on a screening application of SPR”, but acknowledge that this is work is not a screening application when they state later:“Through SPA screening for inhibition of binding of interleukins to receptors, two small molecule inhibitors were found. These were tested in the SPR assay with immobilised receptor”. Clearly, SPA—and not SPR—was used in this study to screen for compounds. This is presumably because the authors had not found a way of applying interfacial optical assays such as SPR for the task. One reason for this may be that the affinity of the interaction measured was relatively high (approximately 2 niM) and thus at least several hours would be required for the ligand to clear from the sensor chip before a further round of screening could be performed.

Taremi et al, (BIAjournal, 1996, 3(1):21) disclose the use of SPR to screen for small molecules which inhibit the binding of human interleukin 4 (IL-4) and its receptor (IL-4R). It is stated that up to 2000 compounds can be tested in less than 24 hours. However, this figure appears to be based on using mixtures of test compounds, especially as the affinity of the interaction between IL-4 and IL-4R is relatively high, meaning that several hours would be required for IL-4R to clear from the immobilised IL-4 on the chip before a further round of screening could be performed.

As mentioned above, it is immaterial for the method of the present invention whether binding of a compound, because of its small size, is within the detection limits of the interfacial optical assay or not. This is because receptor-ligand binding is monitored after exposure to the candidate compound. On the one hand, if the ligand is exposed to the receptor after exposure to the candidate compound and the characteristic, transient increase in refractive index is produced, this shows that the receptor has bound the ligand, and thus that the compound has not bound the ligand in a manner so as to inhibit or prevent the ligand-receptor interaction. On the other hand, if no signal, or a significantly reduced signal, is observed wvhen the ligand is contacted with the receptor after exposure to the candidate compound, then the indication is that the compound has blocked or inhibited the interaction between the ligand and the receptor.

It is also immaterial for the method of the present invention if a candidate compound produces irrelevant “noise” signals by binding with high stability, perhaps even irreversibly, to sites on the receptor that do not interfere with binding to the ligand. Such binding will increase the baseline signal, but will not prevent the detection of the interaction with the ligand, since the characteristic transient increase in signal caused by ligand binding can be detected “on top” of the increased baseline signal.

It is a feature of fast-kinetic interactions, i.e. those having relatively high off rates, that the ligand will very quickly be washed from the receptor. Thus, because the interfacial optical assay allows the binding event to be recorded in real time, the innmobilised receptor can be re-used for other binding events very soon after verifying that the ligand binds to the receptor. This allows individual binding events to be performed sequentially, i.e. separated in time. The advantage of this is that the same immobilised receptor can be used over and over again to test whether many compounds have inhibitory effects on the receptor-binding interaction. This is economical in terms of the (a) work required to immobilise the receptor and (b) the actual amount of receptor used in the assay, compared to an assay in which binding events are separated in individual reaction chambers (as is the case for SPA for example). Furthermore, the ligand can be recovered after being washed from the receptor (in step a) for example) and re-used for further assays.

In addition, the feature that the binding events are separated in time allows buffer conditions, when present, to be identical every time that the availability of the receptor binding site is tested with the ligand. If a test compound and the ligand are present at the same time (as is the case in SPA), changes in buffer conditions or contaminants carried with the compound could affect the ability of the ligand to bind, leading to false indications of inhibition by the compound. Such effects are particularly likely to occur when the receptor-ligand interaction in question is low affinity, mainly because optimal binding conditions are required for the detection of such interactions. A contaminant may denature a ligand, even only partially, or alter the pH, thus preventing it from being able to bind to its receptor.

Optional step c) may be not optional and may be used to ensure that the method of the invention is likely to only identify compounds which act as inhibitors through relatively stable interactions with the immobilised receptor. Compounds which bind only transiently to the specific interaction site on the immobilised receptor, potentially preventing binding of the ligand, can be washed away before testing with the ligand is performed. Indeed, the stringency with which washing is carried out can be adjusted as desired. Examples of parameters which can be adjusted to alter the stringency of washing are known to the skilled person and include:the amount of time that the washing buffer is passed over the immobilised receptor; the concentration of salt in, or the pH of, the washing buffer; additives to the buffer, such as urea, detergents (e.g. Tween, NP40) and other proteins (e.g. bovine serum albumin); and so on. This allows the screening conditions to be selected so as to apply more or less stringent selection criteria to the length of the half-life with which individual compounds bind to the immobilised ligand.

Thus, step c) may be used to eliminate many unsuitable compound candidates which would give positive results in competitive assay types where the compound and receptor are present at the same time (such as in SPA). Particularly where interactions with fast binding kinetics are concerned, these compounds with relatively low binding stability would be able to compete out the binding of the receptor in a competitive assay. However, such compounds would rarely possess sufficient affinity to be effective in vivo for two reasons. Firstly, their specificity would, in all likelihood, be insufficient for a high enough proportion to find the right targets in the human body. Secondly, even if they were able repeatedly to bind to the relevant ligand in vivo, they may not be able to compete as efficiently with receptor-ligand interactions as in the assay, because, in vivo, cell-surface receptor-ligand interactions would ofen be multivalent, greatly enhancing the avidity of these interactions and thereby their ability to compete with the monomeric inhibitor-ligand interactions.

The method of the present invention also lends itself readily to automation owing to the sequential nature in which the various reagents (candidate compound, ligand, washing material, etc) are applied and to the manner in which the binding events are detected in real time. For example, existing Biacore SPR machines have a robot arm for the application of such reagents.

The method of the invention may be made more efficient by contacting the receptor with a sample comprising a predetermined plurality of candidate compounds in step b). If the sample causes inhibition of receptor-ligand binding, the compound(s) responsible for this inhibition can be identified by assaying each individual compound of the sample. In addition, the method can be made more efficient by mixing the compound(s) with the ligand prior to exposure to the receptor.

Optional step a) provides a control step to test that the receptor binds the ligand. The present invention may include additional control binding experiments. For example, the method may include one or more “parallel” controls, whereby the effect of a candidate compound on the binding of one or more control receptors to control ligands specific for those receptors is monitored. Such control(s) test whether the compound specifically inhibits the receptor-ligand binding. Thus, the method may comprise the additional steps of:a1) optionally contacting a control receptor with a control ligand, the control receptor being imrnobilised so that binding of the control ligand therewith can be detected in an interfacial optical assay, detecting by interfacial optical assay the binding of the control ligand to the control receptor, and washing the control ligand from control the receptor; b1) contacting the n^{th} candidate compound with the immobilised control receptor; c1) optionally washing the control receptor at the predetermined stringency; d1) contacting the control receptor with the control ligand, and detecting by interfacial optical assay whether or not the control ligand or control ligand-compound complex has bound to the control receptor or control receptor-compound complex.

Steps b1) and c1) may be carried out simultaneously with steps b) and c) respectively.

Steps a1) and d1) may be carried out before or after steps a) and d) respectively.

In addition to providing a control, the or each “parallel” assay may itself also be a screen for a compound which inhibits the binding of the control receptor and control ligand, in this case, the assay to which it is run in parallel providing a corresponding control. For example, if MHC class I-peptide and MHC class II-peptide complexes are inunobilised, then CD4 can be used as a control to show specific inhibition of binding of CD8 to MHC class I-peptide and CD8 can be used as a control to show specific inhibition of binding of CD4 to MHC class I-peptide. In fact, the only limitation on the number of these parallel screening/control assays is the number of receptors which can be immobilised and monitored in an interfacial optical assay.

Commercial instruments like the Biacore 2000™ or the Biacore 3000™ provide the option of using up to four sensor cells connected in series. This means that, in many cases, a compound library can be screened for the presence of inhibitors of up to four individual ligand-receptor interactions in a single screening run. The “extra” flowcells can be used for a variety of control binding experiments without introducing any need for increasing the amount of candidate compound used or the amount of time consumed for the screening. This means that the quality and strictness of the screening can be easily increased within a single step protocol. For instance, an extra flowcell could be used for duplication of the assay for the candidate compound, ensuring that the results obtained from the twvo cells are consistent and reproducible. One or two other flowcells could be used for assessing the binding to other immobilised ligands that constitute appropriate controls. In contrast, screening procedures according to which individual compounds are tested in individual reaction chambers, e.g. SPA screening, would require an extra set of wells to be used for each control reaction to be performed, significantly increasing the amount of compound and effort consumed in the screening process. In effect, this means that such screening procedures usually are performed in several stages rather than including control experiments in the first screening.

When the method of the present invention uses SPR to detect ligand-receptor binding, the ligand or receptor must be immobilised on the sensor surfaces. A number of different strategies exist for immobilisation of soluble proteins on the surface of BIAcore surface plasmon resonance chips. The most commonly used are shown in FIG. 1 of the accompanying drawings and are briefly summarised below.

The most frequently used technique is amine coupling, whereby amine groups on the protein surface are coupled to the carboxymethyl group of a CM-5 chip. The chemistry involved in amine coupling of proteins is shown in FIG. 1. The carboxymethyl group is modified using EDC (1-ethyl-3-(3-dimethylaminopropyl)-carbodiimide) and NHS (N-hydroxysuccinimide) which activates the group for reaction with amine groups such as those of lysine residues on protein surfaces.

An alternative coupling method is streptavidin-biotin coupling. In this, streptavidin is immobilised by amine coupling as above. Proteins may be modified to contain biotin using NHS-biotin which will react with amine groups on the surface of proteins by amine coupling. Alternatively, proteins may be engineered to contain a specific biotinylation sequence which is recognised by the bacterial enzyme, BirA (Barker & Campbell, J Mol Biol 146(4):451-67(1981); Barker & Campbell J Mol Biol 146(4): 469-92 (1981); Howard et al Gene 35(3):321-31 (1985); O'Callaghan et al. Anal Biochem 266(1):9-15 (1999); Schatz Biotechnology N Y11(10):1138-43 (1993)). If the protein is expressed in a soluble form in E. coli, these proteins will be biotinylated by the host cell's native BirA enzyme. Alternatively, if another host organism is used or if the protein is expressed in inclusion bodies and refolded in vitro, the protein may be biotinylated in vitro using purified enzyme, Mg²⁺-ATP and biotin. Biotinylated proteins may simply be flowed over the flow-cell containing the immobilised streptavidin to give effective coupling of the biotinylated protein to the flow-cell surface.

Other methods for covalently coupling proteins to CM-5 chip surfaces include thiol, and aldehyde coupling (see FIG. 1), but these are not preferred methods for the proteins of the immune system because of the success of aniine coupling and streptavidin-biotin coupling. A further description of these methods is given in the BIAapplications Handbook (Perkin-Elner, Applied Biosystems). Oligo-histidine—NTA (nickel-nitrilotriacetate) coupling uses a BIAcore NTA-derivatised chip (available from Perkin-Elmer, Applied Biosystems). An oligo-histidine tag (often his₆) may be engineered onto the protein at either terminus and coupling simply involves flowing the his-tagged protein over the flow-cell surface. HQwever, this coupling method has the disadvantage that the affinity of the oligo-histidine-tagged protein for the NTA is usually rather lower than that of a biotin-modified protein for streptavidin and therefore the immobilised protein often slowly releases from the chip resulting in a downward-sloping baseline.

Examples of the interactions for which the present invention can be used to screen for inhibitors (provided that they have appropriately fast kinetics) include:Pleckstrin homology domains (ras-GRF, PLC, Sos) to G protein βγ-subunits (Sawai et al. Biol. Pharm. Bull. 22:229-33(1999)); SIOOB to C-terminus of p53 (Ca-dependent) (Delphin et al. J. Biol. Chem. 274 (15):10539-44(1999)); Grb2 SH2 domains to monocarboxylic-based phosphotyrosyl mimetics (Burke et al. Bioorg. Med. Chem. Lett. 9 (3):347-52(1999)); Parathyroid hormone (PTH) to PTH receptor (antagonised by megalin) (Hilpert et al. J. Biol. Chem. 274 (9):5620-5(1999)); Interferon γ binding to STATI (Lackmann et al. Growth Factors 16 (1):39-51(1998)); Human interferon γ (HuIFNγ) to HuIFNγ receptor (Michiels et al. Int. J. Biochem. Cell. Biol. 30 (4):505-16(1998)); Antiapoptotic compound CGP 3466 to glyceraldehyde-3-phosphate dehydrogenase (Kragten et al. J. Biol. Chem. 273 (10):5821-8(1998)); C-terminal domain of insulin-like growth factor-I receptor and insulin receptor (Li et al FEBS Lett. 421 (1):45-9(1998)); Rifampicin binding to the human glucocorticoid receptor (Calleja et al. Nat. Med. 4 (1):92-6(1998)); Erythropoietin (EPO) to the EPO receptor (Binnie et al. Protein Expr. Purif. 11 (3):271-8(1997)); (GT)n repetitive DNA tracts binding to RecA protein (Dutreix J. Mol. Biol. 273 (1):105-13(1997)); Soluble interleukin-4 (IL-4) receptor α-chain/Ig-γl fusion protein to IL-4 (Seipelt et al. Biochem. Biophys. Res. Commun. 239 (2):534-42(1997)); Binding of CD45 and PTP1B to substrate inhibited by disodium aurothiomalate (Wang et al. Biochem. Pharmacol. 54 (6):703-11(1997)); Plasminogen activator inhibitor type-i to tissue plasminogen activator (inhibited by monoclonal antibodies) (Bjorquist et al. Biochim. Biophys. Acta 1341 (1):87-98(1997)); S100A1 to the Ca²⁺ release channel (ryanodine receptor) (Treves et al. Biochemistry 36 (38):11496-503(1997)); Murine VEGF-C binding to Flt4 receptor protein tyrosine kinase (Fitz et al. Oncogene 15 (5):613-8(1997)); Staphylococcus aureus clumping factor to fibrinogen (McDevitt et al. Eur. J. Biochem. 247 (1):416-24(1997)); Insulin analogues binding to insulin receptor (Kruse et al. Am. J. Physiol. 272 (6 Pt 1):El 089-98(1997)); SH2 domains of ZAP-70 to the tyrosine-based activation motif 1 sequence of the ζ-subunit of the T-cell receptor (Labadia et al. Arch. Biochem. Biophys. 342 (1):117-25(1997)); Interaction of lipoproteins with heparan sulphate, heparin and lipoprotein. lipase (Lookene et al. Biochemistry 36 (17):5267-75(1997)); Fas (CD95) binding to Fas ligand (Starling et al. J. Exp. Med. 185 (8):1487-92(1997)); Plasma thrombopoietin binding to the c-Mp1 receptor (Fielder et al. Blood 89 (8):2782-8(1997)); Interleukin-6 binding to the-gp130 receptor (blocked by monoclonal antibodies) (Liautard et al. Cytokine 9 (4): 233-41(1997)); Dac g 4 pollen allergen binding to IgE antibody and to monoclonal antibodies (Leduc-Brodard et al. J. Allergy Clin. Immunol. 98 (6 Pt 1):1065-72 (1996)); Interleukin-2 (IL-2) binding to IL-2 receptor (Myszka et al. Protein Sci. 5 (12):2468-78(1996)); Growth arrest-specific gene 6 product to Axl, Sky and Mer receptor tyrosine kinases (Nagata et al J. Biol. Chem. 271 i(47):30022-7(1996)); Neurocan chongroitin sulphate proteoglycan to N-CAM neural cell adhesion molecule (Retzler et al. J. Biol. Chem. 271 (44):27304-10(1996)); Soluble CD21 binding to iC3b and CD23 (Fremeaux-Bacchi et al. Eur. J. Immunol. 26 (7):1497-503(1996)); Annexin I binding to profilin (Alvarez-Martinez et al. Eur. J. Biochem. 238 (3):777-84(1996)); p59fyn binding to Y602 Sek autophosphorylation site (Ellis et al. Oncogene 12 (8):1727-36(1996)); Human growth hormone (hGH) (and variant) binding to hGH-receptor (Andersson et al. Int. J. Protein Res. 47 (4):311-21(1996)); C-terrninal Src kinase (Csk) binding to Lck (Bougeret et al. J. Biol. Chem. 271 (13):7465-72(1996)); Human interleukin 4 (huIL-4) to huIL-4 receptor α-subunit (Taremmi et al. Biochemistry 35 (7):2322-31(1996)); Grb2 binding to Sosl (Sastry et al. Oncogene 11 (6):1107-12(1995)); Soluble CD14 binding to lipopolysaccharide (Juan et al. J. Biol. Chem. 270 (29):17237-42(1995)); Soluble interleukin-2 (IL-2) receptor binding to IL-2 (Wu et al. J. Biol. Chem. 270 (27):16045-51(1995)); Heparin binding to β A4 amyloid precursor protein enhanced by Zn²⁺ (Multhaup et al. J. Mol. Recognit.8 (4):247-57(1995)); Calmodulin-like domains of calpain binding to calpastatin subdomains (Takano et al. FEBS Lett. 362 (1):93-7(1995)); Lck-derived SH2 domain binding to tyrosine-phosphorylated peptides (von Bonin et al. J. Biol. Chem. 269 (52):33035-41(1994)); Collagen-binding stress protein HSP47 binding to collagen (Natsume et al. J. Biol. Chem. 269 (49):31224-8(1994)); Cyclosporin A and analogues binding to cyclophilin (Zeder-Lutz et al. J. Chromatogr. B Biomed. AppI. 662 (2):301-6(1994)); Calmodulin binding to calcineurin-derived peptide (Takano et al. FEBS Lett. 352 (2):247-50(1994)); Tumour necrosis factor a and lymphotoxin binding to p55 TNF receptor (Corcoran et al. Eur. J. Biochem. 223 (3): 831-40(1994)); Grb2 binding to epidermal growth factor receptor (EGFR) (Batzer et al. (Mol., Cell. Biol. 14 (8):5192-2011994)); Human interleukin-5 (hIL-S) binding to soluble hIL-5 receptor (Morton et al. J. Mol. Recognit. 7 (1):47-55(1994)); ETS1 oncoprotein binding to DNA binding site (Fisher et al. Protein Sci. 3 (2):257-66(1994)); Rat CD2 binding to CD48 (van der Merwe et al. EMBO J. 12 (13):4945-54(1993)); α3β1 intergrin homophilic binding (Sriramarao et al. J BioL. Chem. 268 (29):22036-41(1993)).

The method of the present invention finds particular use in screening for compounds which inhibit the interactions such as MHClpeptide complex-T cell receptor (TCR), MHC-CD8 and MHC-CD4 interactions. Thus, the present invention provides a molecule selected from MHC, MHC-peptide complex, T cell receptor, CD8 and CD4 immobilised for use in an interfacial optical assay.

MHC molecules are specialised protein complexes which present short protein fragrnents, known as peptide antigens, for recognition on the cell surface by the cellular arm of the adaptive immune system, and are divided into Class I and Class II. A wide spectrum of cells can present antigen in MHC/peptide complexes, and the cells that have that property are known as antigen presenting cells (APC). The type of cell which presents a particular antigen depends upon how and where the antigen first encounters cells of the inmmune system. APCs include the interdigitating dendritic cells found in large numbers in the T cell areas of the lymph nodes and spleen in large numbers; Langerhans cells in the skin; follicular dendritic cells in B cell areas of the lyrnphoid tissue; monocytes, macrophages and other cells of the monocyte/macrophage lineage; B cells and T cells; and a variety of other cells such as endothelial cells and fibroblasts which are not classical APCs but can act in the manner of an APC.

Specific MHC-peptide complexes are recognised by T cells, recognition being mediated by the T cell receptor (TCR) which consists of an α and a β chain, both of which are anchored in the membrane. In a recombination process similar to that observed for antibody genes, the TCR α and β genes rearrange from Variable, Joining, Diversity and Constant elements creating enormous diversity in the extracellular antigen binding domains (10¹³ to 10¹⁵ different possibilities).

Antibodies and TCRs are the only two types of molecules which recognise antigens in a specific manner. Thus, the TCR is the only receptor specific for particular peptide antigens presented in MHC, such an antigen often being the only sign of an abnormality within a cell.

TCRs are expressed in enormous diversity, each TCR being specific for one or a few MHC-peptide complexes. Contacts between TCR and MEIC-peptide ligands are extremely short-lived, usually with a half-life of less than a second. Adhesion between T cells and target cells presumably TCRIMHC-peptide relies on the employment of multiple TCR/MHC-peptide contacts as well as a number coreceptol-ligand contacts.

T cell activation models attempt to explain how such protein-protein interactions at an interface between T cell and antigen presenting cell (APC,) initiate responses such as killing of a virally infected target cell. The physical properties of TCR-pMHC interactions are included as critical parameters in many of these models. For instance, quantitative changes in TCR dissociation rates have been found to translate into qualitative differences in the biological outcome of receptor engagement, such as full or partial T cell activation, or antagonism (Matsui et al Proc Natl Acad Sci USA 91(26):12862-6 Issn:0027-8424 (1994); Rabinowitz et al Proc Natl Acad Sci USA 93(4):1401-5 Issn:0027-8424(1996); Davis et al Annu. Rev. Immunol. 16:523-544 (1998)).

TCR-peptide/MHC interactions have been shown to have low affinities. Some studies have used Biosensor technology such as Biacore™, which exploits SPR and enables direct affinity and real-time kinetic measurements of protein-protein interactions (Garcia et al Nature 384(6609):577-81 Issn:0028-0836 (1996); Davis et al Annu. Rev. Immunol. 16:523-544 (1998)). However, the receptors studied are either alloreactive TCRs or those which have been raised in response to an artificial immunogen.

When the method of the present invention is used to screen for inhibitors of MHC/peptide complex-T cell receptor (TCR), MHC-CD8 and MHC-CD4 interactions, it is preferred if the respective receptors and ligands are able to be produced in soluble and/or multimeric form.

Soluble Class I MHC/peptide complexes can be obtained by cleaving the molecules of the surface of antigen presenting cells with papain (Bjorlknan et al, J. Mol. Biol. 186:205-210, (1985)). Although this approach provides material for crystallisation, it has in recent years been replaced by individual expression of heavy and light chain in E. coli followed by refolding in the presence of synthetic peptide (Garboczi et al Proc Natl Acad Sci USA 89(8):3429-33 Issn:0027-8424 (1992); Madden et al [published erratum appears in Cell 1994 Jan 28;76(2):following 410]. Cell 75(4):693-708 Issn: 0092-8674 (1993); Garboczi et al J Mol Biol 239(4):581-7 Issn:0022-2836 (1994); Reid et al J Exp Med 184(6):2279-86 (1996); Reid et al FEBS Lett 383(1-2):119-23 (1996); Smith et al Immunity 4(3):215-28 Issn:1074-7613 (1996); Smith etal Immunity 4(3):203-13 Issn:1074-7613 (1996); Gao et al Nature 387(6633):630-4 (1997); Gao et al Prot. Sci. 7:1245-49 (1998)). This approach has several advantages in that a better yield can be obtained at a lower cost, peptide identity can be controlled very accurately, and the final product is more homogeneous. Furthermore, expression of modified heavy or light chain, for instance fused to a protein tag, can be easily performed.

Methods are also known for the formation of Class II MHC/peptide complexes. These may be modified to make them soluble and to include biotinylation tag sequences to enable immobilisation on a streptavidin-modified CM-5 Biacore chip surface. For example, full length DRB1*0401 was expressed on the surface of Drosophila melanogaster Schneider 2 cells under control of the Drosophila metallothionein promoter which was induced by copper sulphate (Hansen et al Tissue Antigens 51(2): 119-28 (1998)). This approach is easily modified to produce soluble MHC class II molecules simply by expressing a truncated version of the protein which contains a biotinylation tag sequence in place of the transmembrane domain. This protein would be secreted in a soluble form instead of bound to the extracellular surface of the cell membrane.

In another report, the α- and β-chains of HLA-DR1 were expressed in E. coli as inclusion bodies and were purified under denaturing conditions separately prior to refolding in vitro (Frayser et al Protein Expr Purif 15:105-14 (1999)). The protein produced was soluble and stable, and bound peptide in the expected manner. It would be very straightforward to modify this procedure to include a biotinylation tag sequence to enable linking of this protein to a Biacore chip.

Increased stability between the ax and P chains of soluble Class II MHC molecules has been achieved by expressing them as fusion proteins. Membrane domains of the HLA-DR2 molecule α- and β-chains (DRA, DRB1*1501 genes) were replaced with leucine zipper domains from c-jun and c-fos (Kalandadze et al. J Biol Chem 271:20156-62 (1996)). Expression was achieved in methyltrophic yeast (Pichia pastoris) using the α-mating factor secretion signal to direct expression to the secretory pathway. This procedure could be easily modified to include a biotinylation tag sequence on one of the protein chains.

The production of soluble T-cell receptor specific for class I and class II MHC-peptide complexes is also known. In WO99/60120 (Willcox et al, Immunity 10:357-365 (1999), Willcox et al, Prot. Sci 8:2418-2423 (1999)), a method for the production of soluble TCR is described in which the extracellular fragments of TCR are expressed separately as fusions to the “leucine zippers” of c-jun -and c-fos and then refolded in vitro. The TCR chains do not form an interchain disulphide bond as they are truncated just prior to the cysteine residue involved in forming that bond in native TCR. Instead the heterodimeric contacts of the α and β chains are supported by the two leucine zipper fragments which mediate heterodimerisation in their native proteins. Alternatively, TCR could be produced in eukaryotic cells according to the methods of Garcia, et al. (Science 274(5285):209-19 (1996) Issn:0036-8075; Nature 384(6609): 577-81 (1996) Issn:0028-0836). However, this is not preferred because the material is extremely expensive to produce.

The method of the present invention may use multimeric T cell receptors, the production of which is disclosed in WO99/60119.

The vast majority of T cells restricted by Class I MHC-peptide complexes also require the engagement of the coreceptor CD8 for activation, while T cells restricted by Class II MHC require the engagement of CD4. The exact function of these coreceptors in T cell activation is not yet entirely clarified, neither are the critical mechanisms and parameters controlling activation. However, both CD8 and CD4 have cytoplasmic domains which are associated with the kinase p56^{Ick} which is involved in the very earliest tyrosine phosphorylation events which characterises T cell activation. CD8 is a dimeric receptor, expressed either in a o(x form or, more commonly, in an αβ form. In the CD8 receptor, only the ox chain is associated with p₅₆^{Ick}.

The peptide-specific recognition of antigen presenting cells by T cells is probably based on the avidity obtained through multiple low-affinity receptoriligand interactions. These involve TCRPMHC-peptide interactions and a number of coreceptoriligand interactions. The CD4 and CD8 coreceptors of class II restricted and class I restricted T cells, respectively, also have the MHC, but not the peptide, as their ligand. However, the epitopes on the class I MHC with which CD8 interacts and the epitopes on class II MHC with which CD4 interacts do not overlap those of TCRs.

This recognition mechanism opens the possibility that peptide-specific recognition of antigen presenting cells can be modulated through inhibition of coreceptor binding. Indeed, it has been demonstrated that soluble, recombinant CD8 derived from the human coreceptor is a potent inhibitor of class I MHC-restricted T cells responses (Sewell et al. Nature Medicine 5:399-404 (1999)).

Expression of soluble CD8 is described, for example, in Gao et al, Prot. Sci. 7:1245-49 (1998).

Recent determinations of the physical parameters controlling binding of TCR and CD8 to MHC, using soluble versions of the receptors, has shown that binding by TCR dominates the recognition event. TCR has significantly higher affinity for MHC than the coreceptors (Willcox, et al. Immunity 10:357-65 (1999); Wyer et al. Immunity 10: 219-225 (1999)).

CD4 is a monomer and there have been a substantial number of reports which describe the production of recombinant soluble CD4. Expression systems used include bacculo virus directed expression in insect cells (Hussey, et al. Nature 331(6151):78-81 1988)); vaccinia virus directed expression in mammalian cells (Berger, et al Proc Natl Acad Sci USA 85(7):2357-61 (1988)); Chinese hamster ovary cells stably transfected with an expression plasmid (Carr, et al. [published erratlun appears in J Biol Chem 265(6):3585 (1990)].” J Biol Chem 264(35):21286-95 (1989); Davis, et aL J Biol Chem 265(18):10410-8 (1990); Allaway, et al. AIDS Res Hum Retrovinses 11(5):533-9 (1995)); and bovine pappiloma virus directed expression in Mouse C-127 cells (Gidlund, et al. Arch Virol 113(3-4):209-19 (1990)). Soluble CD4 has also been produced by expression in E. coli followed by chemical refolding in vitro. Amino acids 1-183, constituting the two N-terminal Ig domains of CD4 were used to inhibit HIV infection of peripheral blood lymphocytes in vitro (Garlick, et al. AIDS Res Hum Retroviruses 6(4):465-79 (1990)). The same effect was demonstrated with a protein expressed in Chinese hamster ovary cells and also constituting the two most N-terminal domains of CD4 fused to IgG2, resulting in a tetrameric form of the soluble protein (Allaway, et al. AIDS Res Hum Retroviritses 11(5):533-9 (1995)). Furthermore, Traunecker, et al Nature 339:68-70 (1989) describe dimeric CD4-IgG molecules and pentamneric CD4-IgM molecules and their application in inhibition of HIV infection in vitro. Bacterial expression was increased by using a T7 based system for expression in bacteria (Kelley, et al. Gene 156(1):33-6 (1995)), and recently, it was reported that amino acids 1-183 of CD4 can also be expressed as a secreted, soluble protein from bacteria, bypassing the need for the refolding step (Osbume, et al. J Immunol Methods 224(1-2):19-24 (1999)).

Attempts to measure the affinity of CD4 for MHC class II molecules have failed since it apparently is too low to be detected reliably by the techniques available. The low affinity prevents the investigation of the ability of chemical compounds to interfere with the CD4/HLA-interaction. Thus, it is preferred that CD4 be modified to allow increased avidity of binding, preferably without inducing changes in the affinity of the interaction with MHC Class I.

CD4 nmay be modified by being multimerised so as to form a multivalent CD4 complex comprising a plurality of monomeric CD4 molecules. The multivalent CD4 complex may be a multimer which may comprise two, three, four or more CD4 monomers.

Multimenrsation may be by means of an multimerisation module which may be attached to or associated with each monomer in complex. It is preferred if the multimerisation module is attached or associated with the C-terminus of each molecule. CD4 may be multimerised as described in Allaway, et al. AIDS Res Hum Retroviriuses 11(5):533-9 (1995) (fusion to IgG2 to form tetramers), or in Traunecker, et al Nature 339:68-70 (1989) (fusion to IgG to form dimers and fusion to IgM to form pentamers).

One example of a multimerisation module is a coiled coil domain, also known as a “leucine zipper”. Leucine zippers are protein modules that mediate protein-protein interactions most commonly between cellular transcription factors (McKnight, Sci Am 264(4) 54-64 (1991)). The name derives from early models in which leucine residues repeated in a heptadic pattern along two aligned alpha helices would interdigitate in the same way the teeth interdigitate in real zippers (reviewed in Landschulz, et al. Science 240(4860):1759-64. (1988)). The heptade repeat motif has been identified h a number of transcription factors such as C/EBP (Descombes, et al. Genes Dev 4(9): 1541-51 (1990)), CREB (Gachon, et al. J Virol 72(10):8332-7 (1998)), C/ATF (Yukawa et al. Brain Res Mol Brain Res 69(1):124-34 (1999)), c-Fos and c-Jun (O'Shea, et al. Science 245(4918):646-8 (1989)), and GCN4. Zippers from these factors have been used to direct the oligomerisation of a number of genetically engineered chimeric proteins (Kim and Hu Mol Microbiol 25(2):311-8 (1997); Zeng, et al. Protein Sci 6(10):2218-26 (1997); Willcox, et al. Immunity 10:357-65 (1999)).

The yeast transcription factor GCN4 was shown to form stable homodimers and the structure responsible for DNA binding and dimerisation were shown to localise within the C-terminal 50 amino acids of the protein (Hope and Struhl Embo J 6(9):2781-4 (I 987,) i X-ray crystallographic studies have revealed that the leucine zipper of G(-N4 folds into a two-stranded parallel coiled coil of alpha helices forming a twisted elliptical cylinder approximately 45 Å long and 30 Å wide (O'Shea, et al. Science 254(5031):539-44 (1991); Rasmussen, et al. Proc Natl Acad Sci USA 88(2):561-4 (1991)). 3.5 amino acid residues are used for each turn of the helix in the coiled coil; thaws residues seven positions apart are stacked exactly on top of each other in the vertical direction. The amino acids therefore occupy one of seven positions on the face of the helix and these are referred to as positions a through g. Stability of the stncture in aqueous solvent is obtained by creating a hydrophobic seam along the interface that is shielded from the surrounding solvent by the neighbouring residues. Thus approximately 1800 Å² of hydrophobic interface surface area is buried in the dimer, 95% of which is provided by residues at positions a, d, e, and g and within the diiner residues at positions a and d are 83% buried. Notably, N₁₆ (see FIG. 9) appears to inflict a steric restriction on the conformation of the native zipper, ensunng that only parallel alignment of the alpha helices takes place. Charged residues K₁₅, E₂₀, E₂₂, and K_{¢}at positions e and g cancel out each other pairwise, allowing the methylene groups of the charged side chains to contribute to the hydrophobicity of the buried seam Apart from A₂₄ (see below) residues at positions b, c, and f extending in the opposite direction from the hydrophobic seam and facing the solvent are all polar or charged.

Leucine zippers can be used to provide oligomers other than dimers. Studies using mutated GCN4 leucine zippers have provided information regarding the ability of different amine acids at different positions for formation of higher order oligomers. Harbury et al. (Science 262 (5138):1401-7 (1993)) induced mutations at the a and d positions (see F igure 9) of the alpha helix of GCN4 and found that zippers made from peptides containing isoleucines at the a-position and leucines at the d-positions would form dimers like the wildtype-derived peptide, while isoleucines at both positions would lead to the fonnation of stable trimers. Substituting with leucines at the a-position in combination with substitutions with isoleucines at the d-position led to the formation of tetramers and characteristically the melting temperatures of the oligomers made from mutated peptides were significantly higher than the wild type zipper. Other combinations including substitutions with valine did not lead to uniform oligomensation in the GCN4-based system (Harbury, et al. Science 262(5138):1401-7 (1993)).

A leucine zipper may be used to provide a CD4 trimer. An antiparallel trimer has been crystallised from a peptide, coil-Ser (see FIG. 9a), (Lovejoy, et al. Science 259(5C99):1 288-93 (1993)). The ability of this peptide to fold into a stable trimeric conformation put a focus on the role of the residues at positions e and g of the individual helices. In theory, the opposite charges of opposing glutamate and lysine residues should cancel out the electrostatic repulsion between the opposed helices and favour parallel alignment. The observed arrangement supposedly was made possible by a rolding the tnrmer at pH 5.0 at which some protonation of glutamate residues allowed formation of stabilising hydrogen bonds between opposed glutamate residues. Based on this observation, Boice et al. (Biochemistry 35(46):14480-5 (1996)) designed a modified coli-Ser peptide, coil-VₐL_{d} (see FIG. 8), with valines at all a-positions and leucines at all d-positions that formed stable parallel trimers. The free energy of stabilisation for this coiled coil was determined to be −18.4 kcal mol⁻¹ and the ΔCₚ of denaturation to be 8.6 cal deg⁻¹ mol⁻¹ residue⁻¹. In FIG. 9a, the different oligomerisation motifs mentioned have been sampled and aligned.

The multimerisation module may comprise a multivalent linker molecule such as avidin, streptavidin or extravidin. Thus, CD4 may be multimerised by engineering onto CD4 so-called “biotinylation tags” (Schatz, Biotechnology NY 11(10):1138-43 (1993) I. Biotinylation of CD4 enables tetramerisation via tetravalent streptavidin (Altman, et al. Science 274(5284):94-6 (1996)) binding four monomeric biotinylated CD4 fusion proteins. The bacterial protein BirA has been shown to transfer biotin to proteins labelled with the tag indicated in FIG. 9b (O'Callaghan, et al. Anal Biochem 266(1):9-15 (1999); Altman, et al. Science 274(5284):94-6 (1996); Schatz, Biotechnology NY 11(10):1138-43 (1993)).

The different designs of CD4 oligomerisation fusion proteins are schematically shown in FIG. 10. In the general design, a hinge/stalk region is introduced between the extracellular domain of CD4 and the oligomerisation domain. This domain is completely synthetic but is designed so that it introduces motifs that are known to disrupt alpha helix formation and induce free rotation around so called proline/glycine hinges. Polar serine residues are included into the domain in order to increase hydrophilicity of the stalk. The stalk region is followed by one of the oligomerisation domains shown in FIG. 10 before the protein is terminated.

The individual interactions of the receptors (CD4 and CD8) with MHC are very short-lived at physiological temperature, i.e. about 37° C. An approximate figure for the half-life of a TCR-MHC/peptide interaction, measured with a human TCR specific for the influenza virus “matrix” peptide presented by HLA-A*0201 (HLA-A2), is 0.7 seconds. The half-life of the CD8αα:interaction with this MHC/peptide complex is less than 0.01 seconds, or at least 18 times faster.

The techniques discussed above to increase the avidity of CD4 binding may be equally used to increase the avidity of CD8 binding. In addition, the method of the present invention may use soluble CD8 produced as described in WO99/21576. The avidity of MHC-peptide complex binding may be increased by multimerising this complex, and such multimers may be used in the present invention. WO96/26962 describes a technique for producing MHC-peptide complex tetramers. The higher avidity of the multimeric interaction provides a dramatically longer half-life for the molecules binding to a T cell than would be obtained with binding of a monomeric peptide-MHC complex. The tetrameric peptide-MHC complex is made with synthetic peptide, β2microglobulin (usually expressed in E. coli), and soluble MHC heavy chain (also expressed in E. coli). The MHC heavy chain is truncated at the start of the transmembrane domain and the transmembrane domain is replaced with a protein tag constituting a recognition sequence for the bacterial modifying enzyme BirA. Bir A catalyses the biotinylation of a lysine residue in a somewhat redundant recognition sequence; however, the specificity is high enough to ensure that the vast majority of protein will be biotinylated only on the specific position on the tag. The biotinylated protein can then be covalently linked to avidin, streptavidin or extravidin, each of which has four binding sites for biotin, resulting in a tetrameric molecule of peptide-MHC complexes.

One method in accordance with the present invention will now be described with reference to FIG. 2 of the accompanying drawings. Referring to FIG. 2a, three sensor cells 1, 2 and 3 are serially connected in the direction of the buffer flow (shown by the arrows). The respective readouts on the SPR instrument are shown below each sensor cell. Sensor cells 1 and 2 have soluble test ligand A immobilised therein, and sensor cell 2 has a soluble control ligand B immobilised therein.

In FIG. 2b, a soluble receptor C which binds specifically to test ligand A is passed through the sensor cells 1-3. It can be seen from the SPR readouts that, as expected, receptor C binds to test ligand A in cells 1 and 3, but not to control ligand B in cell 2. The interaction between test ligand A and receptor C is sufficiently short-lived that binding is only detected while, and very shortly after receptor A is passed over the relevant biosensor surface.

Next, referring to FIG. 2c, a control soluble receptor D is passed through cells 1-3. As expected, control receptor D binds to control ligand B in cell 2, but not to test ligand A in cells 1 and 2.

In the next step (FIG. 2d), a compound E from a compound library is passed through the cells. The compound E binds to test ligand A in cell 1, but not to control ligand B in cell 2. Flow of the compound E through cell 3 is prevented as this cell is retained for subsequent control purposes. In the figure, it is assumed that the compound E is smaller than receptor C and therefore produces a smaller readout from the biosensor in cell 1 than in FIG. 2b. The size of the signal from the binding of the compound and whether this can be detected or not is immaterial. Because SPR detects mass changes on the sensor surface, additive binding is equally well detected and therefore subsequent binding of test receptor C, or the absence of this is the important indicator in the method.

In FIG. 2e, the receptor C is again passed through cells 1-3, as in the step illustrated in FIG. 2b. However, now the receptor C is not able to bind to test ligand A in cell 1 because of the binding of compound E. It is of note that the binding of compound E to test ligand A has a half-life sufficient to remain bound during this step. If the half-life was shorter, compound E would have been washed away in the buffer. Thus, the method enables compounds to be selected which have a predetermined minimum half-life, according to the stringency of the washing step.

The final step is shown in FIG. 2f, which is a control step to show that compound E is a specific inhibitor of the binding of test ligand A to receptor C, i.e. to show that compound E does not block the binding of control receptor D to control ligand B. Control receptor D is passed through cells 1-3, and binds only in cell 2, confirming that compound E has had no effect on the binding of control receptor D to control ligand B.

The Biacore 2000™ SPR system (or the newer Biacore 3000™ SPR system) includes a programmable robot arm which collects pre-prepared samples and delivers them for injection over the chip (sensor) surfaces. The technology used to link the ligands to the flow-cell surfaces will depend upon the molecules employed in the assay, but would typically involve amine-coupling of streptavidin to the flow-cell surface followed by linking of a biotin-modified ligand by simply flowing this over the flow-cell.

Following a round of detections, such as the one outlined above with reference to FIG. 2, it is possible to continue to attempt to detect inhibition by other compounds until some irreversible inhibition is detected. At this point, further data will be compromised by the binding of the inhibitor to the immobilised ligand on the flow-cell surface and a new set of flow-cells will have to be modified to assay further compounds.

It is possible for a sample comprising a predetermined plurality of candidate compounds to be screened for the presence of an inhibitor in a first step. If the present of an inhibitor for a particular ligand-receptor interaction is detected in that sample, then it is possible to fractionate the sample using, for example, chromatographic separation. The separated fractions can be tested in the same manner as outlined above. Any fraction showing inhibition can then be fractionate further until the specific compound within the sample responsible for inhibition is isolated. The compound can then be purified to a larger scale.

The method shown in FIG. 2 uses three biosensor surfaces which enhances the quality (strictness) of the screening that is perform-ed. Several biosensor surfaces, typically between two and four, can be serially connected so that the flow of the non-immobilised interaction partner can be directed, in turn, over sensors with the specific immobilised interaction partner and suitable immobilised control proteins. This allows verification of the specific nature of the interaction which is employed in the assay. If the interaction between two proteins is detected specifically then it follows that specific blocking of the interaction by a separate compound can also be detected The way to do this is to flow the compound, or group of compounds, in question over the specific and control interaction partners, each immobilised in their biosensor compartment, before the non-immobilised interaction partner is passed over the same surfaces 1 see FIGS. 2a-f) Binding of the test compound to the specific interaction partner may, or may not (more likely), itself be detected by a signal from the biosensor (FIG. 2d). However, this is unimportant since its ability to block specifically the interaction site for the non-immobilised interaction partner is demonstrated by the decrease or lack of signal when the soluble receptor is subsequently passed over the biosensor surfaces (FIG. 2e).

In the following are described methods in accordance with the invention using soluble forms of certain of the proteins involved in evoking cellular immune responses for the purposes of testing or identifying compounds with clinical potential. The interactions of these molecules can be measured in real time using a surface plasmon resonance biosensor for instance the Biacore 2000™ system or the Biacore ₃₀₀₀™ system. As reported (Willcox, et al. Immunity 10:357-65 (1999); Wyer et al. Immunity 10:219-225 (1999)). the binding assays are highly accurate, fast, and convenient to perform and, using the protein components produced as described, provide extremely reliable readouts for these highly transient binding events.

In general, compounds that bind specifically to proteins involved in cellular regulation, for example receptors or ligands, have the potential for a wide range of therapeutic applications. The cellular immune system, being directly involved in a wide range of disease-related reactions, is an obvious target for therapeutic modulation by small compounds. Many compounds which bind to receptor or ligand proteins will have direct potential as immune inhibitors by preventing the normal cell signaling pathways being activated. Indeed, the sensitivity of the cellular immune system makes it highly susceptible to inhibition (Klenerman, et al. Nature 369(6479):403-7 (1994); Sette, et al. Annu Rev Immunol 12:413-31 Issn:0732-0582 (1994); Sewell et al. Nature Medicine 5:399-404 (1999)). Furthermore, compounds that bind specifically to a cell surface protein also have potential for a number of other applications, since they can be used to target a subset of cells in the body. This characteristic can be used to carry other compounds to such cells, opening possibilities for a wide range of applications in diagnostics, imaging and in vivo drug delivery.

A. Identification of compounds with the ability to block or inhibit the interaction of a particular peptide antigen-HLA combination with TCRs.

FIG. 3 outlines a method for using SPR detection of TCR-MHC/peptide interactions to test, or screen for, compounds that inhibit or block the MHC/peptide surface for TCR binding. The method has the same steps as the method described with reference to FIG. 1, the particular molecules being as follows:

Test ligand A=MHC/peptide complex for which a compound with binding specificity is sought.

Control ligand B=MHC/peptide complex with identical MHIC but a different peptide.

Test receptor C=TCR which recognises test ligand A

Control receptor D=TCR which recognises control ligand B

Test compound E=test compound

The two MHC/peptide complexes A and B with identical MH.EC proteins but presenting different peptide antigens can be produced as soluble molecules according to one of the methods described ((Garboczi et al Proc Natl Acad Sci USA 89(8):3429-33 Issn: 0027-8424 (1992); Madden et al [published erratum appears in Cell 1994 Jan 28;76(2):following 410]. Cell 75(4):693-708 Issn:0092-8674 (1993); Garboczi et al J Mol Biol 239(4):581-7 Issn:0022-2836 (1994); Reid et al J Exp Med 184(6):2279-86 (1996); Reid et al FEBS Lett 383(1-2):119-23 (1996); Smith et al Immunity 4(3): 215-28 Issn:1074-7613 (1996); Smith et al Immunity 4(3):203-13 Issn:1074-7613 (1996); Gao et al Nature 387(6633):630-4 (1997); Gao et al Prot. Sci. 7:1245-49 (1998); Kalandadze, et al. J Biol Chem 271:20156-62 (1996); Hansen, et al. Tissue Antigens 51(2):119-28 (1998); Frayser et al. Protein Expr Purif 15:105-14 (1999)), and immobilised in the respective sensor cells.

Soluble TCRs can be produced as described in. WO99/60119 and WO99/60120 (Willcox et al, Immunity 10:357-365 (1999), Willcox et al, Prot. Sci 8:2418-2423 (1999)).

Referring to FIG. 3d, if the test sample flowed over sensor cells 1 and 2 contains a compound E that binds with high stability to the MHC/peptide complex A in sensor cell 1, a higher constitutive level of readout may be observed if the compound E is of sufficient size for a change in mass to be detected. However, whether the compound E itself produces a sufficient change in mass for detection is immaterial, since the presence and specificity of the MHC/peptide-compound interaction is demonstrated by subsequent testing with the relevant and control TCRs (FIGS. 3e and 3f, respectively). With the compound E bound to the MHC/peptide complex A in sensor cell 1, the TCR C cannot bind but can still bind in sensor cell 3, which was not exposed to the compound test sample E (FIG. 3). This serves to demonstrate that the TCR C is functional and that lack of binding to sensor cell 1 is caused by the compound E. Normal binding of TCR B in sensor cell 2 demonstrates that the compound E has not bound here and is specific for the peptide of complex A (FIG. 3f).

It is important to note that the low affinities and fast kinetics of the TCR-MHC/peptide interaction are crucial to this screening strategy. Only because of the fast off-rates of TCR-MHC/peptide interactions (Willcox, et al. Immunity 10:357-65 (1999)), is binding detected only while the samples of soluble TCRs are flowed over the sensor surfaces. The MHC/peptide complex is left free to be bound by another compound almost immediately after the soluble TCR sample has flowed through the sensor cell.

The method could be modified by using four sensor cells instead of three. In this case, simultaneous screening could be performed for compounds with affinity for either MHC/peptide complex A or B. The sensor cell 4 would have MHC/peptide complex B immobilised therein and serve the equivalent control purposes for binding to sensor cell 2 as sensor cell 3 does for sensor cell 1. The two TCRs C and D would serve as specificity controls for each other.

The human body has the capacity to produce huge repertoires of two types of antigen receptors, antibodies (Ab's) and TCRs. Ab's and TCRs constitute the basis for adaptive immunity. Ab's bind suitable epitopes through interactions that are usually characterised by relatively high affinity. In contrast, TCR binding to MHC/peptide is characterised by low affinity, with recognition of the antigen presenting cell by the T cell relying on higher avidity accomplished through multiple interactions. This also appears to be the case for many other interactions between cell-surface proteins involved in regulating the cellular immune system (Davis, et al. Annu. Rev. Immunol. 16:523-544 (1998); Davis, et al. Imm. Rev. 163:217-36 (1998)).

Three features of TCR recognition of MHC/peptide makes this class of interactions particularly attractive for interference by small compounds:

TCRs are specific for cell-surface antigens. Thus, if a small compound is found that only binds to a particular MHC/peptide complex and interferes with TCR binding, then this compound must be peptide antigen-specific. Because humans of the same MHC type usually present the same peptide antigen when suffering from a particular disease (be it viral infection, cancer or immune disorder), such a compound will have specificity for the disease-relevant cells in the affected population of the relevant MHC type.

Because of the relatively low affinity of TCR-MHC/peptide interactions, there is a considerable range of affinities within which compounds with MHC/peptide binding specificity would have higher affinities than TCRs. There is thus considerable scope for identifying compounds that would be suitable as T cell inhibitors by means of competitive binding to MHC/peptide complexes.

TCR signaling is exquisitely sensitive to interference, as demonstrated by “T cell antagonism” in which subtly modified peptide ligands display great potency for preventing full signaling activation in response to the “normal” peptide antigen (Klenerman, et al. Eur J Immunol 25(7):1927-31 Issn:0014-2980. (1995); Sloan Lancaster & Allen Curr Opin Immunol 7(1):103-9 Issn:0952-7915 (1995); Sloan Lancaster & Allen Annu Rev Immunol 14:1-27 Issn:0732-0582 (1996); Sewell et al. Eur J Immunol 27(9):2323-9 (1997); Purbhoo et al Proc. Natl. Acad. Sci. USA 95:4527-4532 (1998)). Thus, T cell responses may also be sensitive to interference by other means, for instance, interference by competitive ligand binding by small compounds.

These considerations make it likely that TCR-MHClpeptide interactions are suitable targets for T cell inhibition with small compounds. In humans, this type of therapy would be useful to prevent unwanted T cell responses, for example those causing autoimmune diseases or graft rejection following transplant operations. In particular, MHC/peptide-specific compounds are likely to be substantially more specific in their immune inhibitory effect than currently applied treatments for such conditions.

Compounds specific for peptide antigens presented on the cell surface as a consequence of, for example, viral infections or cancerous transformation of body cells also have therapeutic potential, albeit for different applications than immune inhibition. Such compounds could for instance be used as carriers of other, cytotoxic, compounds. Such compounds are well-known to the skilled person and include cis-platin, cytotoxic alkaloids, calcein acetoxymethyldester (Johsson et al, Eur. J Cancer. 32a 883-7 (1996)), and 5-fluoroorotate (Heath et al, FEBS Lett. 187:73-5 (1985)). This strategy could be applied for highly specific drug delivery strategies in the human body. In some cases, most notably in cancer tumours, not all malignant cells present antigen, and it may be desirable to affect a local area rather than only the subset of cells that are antigen presenting. Cytotoxic T cells do not have this capacity but, depending on the therapeutic agent which is carried, it may be possible to achieve such an effect by in vivo drug delivery mediated by a small peptide antigen-specific compound.

In addition, peptide-specific compounds could have potential in diagnostics, for instance by coupling it to a biosensor, or in in vivo imaging by coupling it to a suitable detectable reagent. Such reagents are well-known to the skilled person and include Gd-containing liposomes (Trubetskoy et al, Magn. Reson, Imaging 13:31-7 (1995)) and MION 46 (Shen et al, Bioconjug. Chem. 7:311-6 (1996)).

It is to be noted that the above method can only be applied to diseases for which the relevant peptide antigen and its HLA restriction have been identified. However, there is a considerable number of important diseases for which this is already the case and more disease-relevant peptide antigens are being identified all the time. Examples of diseases for which the relevant peptide antigen and its HLA restriction have been identified include:the MAGE-1 antigen for hepatocellular carcinomas (Yamashita et al, Hepatology 24:1437-1440 (1996)); the MAGE-1, BAGE and BAGE-I antigens for ovarian carcinomas (Russo et al, Int. J. Cancer, 67:457-460 (1996)); the BAGE antigen for melanoma (Boel et al, Immunity, 2:167-175 (1995)); T cell epitopes from glutaric acid decarboxylase for insulin-dependent diabetes mellitus (IDDM) (Endl et al, Arthritis Rheum. 40:1115-1125 (1997)); myelin basic T cell epitopes for multiple sclerosis (Wuncherpfenning et al, J. Clin. Invest. 100(5):1114-1122(1997)); Borrelia burgdorferi outer surface protein A (OspA) T cell epitopes for Lyme disease (Karnradt et al, Infect. Immun. 64(4):1284-1289 (1996)); HIV-1 and HIV-2 cytotoxic T cell epitope for HIV (Nixon et al, AIDS, 4(9):841-845 (1990)); Influenza nucleoprotein T cell epitope for influenza virus (Bowness et al Eur. J. Immunol. 24(10):2457-63 (1994)); and ESAT-6 T cell epitopes for Mycobacterium tuberculosis (Ravn et al, J. Infect. Dis. 179(3):637-45 (1999)).

### EXAMPLE A1

Use ofBIAcore bionzolecular interaction analysis as a methodfor screening compounds to inhibit the interaction between soluble T cell receptor and peptide MHC complex

BIAcore 3000™ urface plasmon resonance technology was used to testing a compound library for small molecules which inhibit the interaction between T cell receptors (TCRs) and their cognate peptide-MHC molecules.

The JM22 and A6 soluble T cell receptors, specific for the influenza matrix peptide-HLA-A2 complex and tax 11-19 peptide-HLA-A2 complex respectively, were prepared as described in WO99/60120A. Peptide-HLA-A2 complex was prepared as described in Garboczi et al, PNAS 89:3429-3433 (1992).

sTCRs were transferred into HBSE buffer (10 mM HEPES pH 7.4, 150 mM NaCl, 3 mM EDTA) using gel filtration chromatography (Pharmacia 26/60 Superdex 200 PG column) and concentrated to using a Millipore centriprep concentrator (10 kDa cut-off). A6 sTCR and JM22 sTCR were prepared to a concentration of 2.0 mg/ml and 1.3 mg/ml, respectively, prior to screening.

A CM-5 sensor chip was docked onto the BLAcore ₃₀₀₀™. Streptavidin was coupled to the carboxymethyl surface using standard amine coupling. The chip surface was activated with 0.2M EDAC/0.05M NHS, followed by binding of streptavidin (0.25 mg/ml in 10 mM sodium acetate pH 5.0) and saturation of unoccupied site with 1 M ethylenediamine.

Biotinylated HLA-A2 (complexed with either the influenza matrix peptide or the tax 11-19 peptide) and a control protein (biotinylated A6 sTCR was used as the immobilised control protein in these experiments) were immobilised on the streptavidin-coated surface (test flow cell and control flow cell respectively) until a response of approximately 1000-5000 RU was observed.

Mixtures of library compounds were solubilised in DMSO to a concentration of 0.5 mg/ml, then diluted into BIAcore buffer to a concentration of 50 μg/ml of each compound in the mixture, including a total of 8% DMSO. This was then diluted IOx in BIAcore buffer to make a final working solution (5 pg/ml per compound, 0.8% DMSO).

BLAcore 3000™ was run at a flow rate of 10 μl/min using BLAcore buffer so that the immobilised peptide-HLA-A2 complex was exposed to sTCR and mixture of compounds. BIAcore COINJECT program was used during screening so that the exposure of the chip surface to the sTCR followed on directly from the exposure to the compounds. Data were recorded automatically and were analysed using BIAevaluation software. sTCR specific responses were calculated taking account of any variance in the baseline between the two cells.

FIG. 4 shows the response from binding of JM22 sTCR to flu-HLA-A2. The response from the flow cell coated with flu-HLA-A2 is shown as the solid line and the control flow cell as a dotted line. Shown are the initial control injection of sTCR and the first round of screening (compounds A1-H1). No inhibition of binding was observed. Overall, the loss of signal was 9.3%, although after 10 mixtures had been passed over the flow cell, a loss of 10.2% was observed

Similar results were obtained for binding of A6 sTCR to tax-HLA-A2. (data not shown). No inhibition of binding was observed after any of the compound mixtures tried. Overall, the loss of signal over the course of the experiment was 33%.

B. Identification of compounds with the ability to block or inhibit a particular peptide HLA molecule for interactions with TCRs, irrespective of the peptide antigen presented.

FIG. 5 outlines a strategy for using SPR detection of TCR-MHC/peptide interactions to test, or screen for, compounds that inhibit or block the surface of a particular HLA molecule for TCR binding, irrespective of the peptide antigen specificity. The method has similar steps to the method described with reference to FIG. 1, the particular molecules being as follows:

Test ligand A=MHC for which a compound with binding specificity is sought with a first peptide.

Control ligand B=different MHC with a third peptide.

Test receptor C=TCR which recognises test ligand A

Control receptor D TCR which recognises control ligand B

Test compound E=test compound

Test ligand F=MHC for which a compound with binding specificity is sought with a second peptide.

Test receptor G=TCR which recognises test ligand F

Thus, the method uses three MHC/peptide combinations, two of which have the same HLA molecule and all of which have different peptide antigens, as well as TCRs specific for each HLA/peptide combination. The MHC/peptide complexes and TCRs are produced as soluble molecules as described before.

The MHC molecule for which a compound with binding specificity is sought is immobilised in sensor cells 1, 2 and 4 (with first peptide antigen (A) in sensor cells 1 and 4, and third peptide antigen (B) in sensor cell 2—see FIG. 5a). The control MHC complex B, which is to serve as control for the specificity of the compound that is being sought, is similarly immobilised in sensor cell 3. When buffer is caused to flow over the sensor cells, the SPR readout is “flat”, indicating no changes in mass on any of the sensor surfaces.

In order to ensure that both the imrnobilised MHC/peptide complexes and the soluble TCRs that recognise them are active, samples of the TCRs (C, D and G) are passed over the sensor cell surfaces (FIGS. 5b-d). The two TCRs (C and G) specific for the HLA molecule for which a compound with binding specificity is sought (A and F) produce signals in sensor cells 1 and 4 and in sensor cell 2, respectively. These TCRs do not produce a signal in sensor cell 3 as they do not recognise the MHC/peptide in this cell (B). Similarly, the control TCR D produces a signal in sensor cell 3 but not in sensor cells 1, 2 and 4 (FIG. 5d). These readouts serve to demonstrate that the soluble proteins employed are functional and specific.

The compound sample to be tested is passed through sensor cells 1, 2 and 3, but not through sensor cell 4 which is retained for subsequent control purposes (FIG. 5F). If the test sample contains a compound E that binds with high stability to the MHC molecule in sensor cells 1 and 2, higher constitutive levels of signal may be observed if the compound is of sufficient size for a change in mass to be detected (FIG. 5f). However, whether the compound E itself produces a sufficient change in mass for detection is immaterial since the presence and specificity of the MUC molecule-compound interaction is demonstrated by subsequent testing with the relevant and control TCRs (FIGS. 5f-g and h, respectively). With the compound E bound to the MHC/peptide complex A in sensor cell 1, TCR A cannot bind but can still bind in sensor cell 4, which was not exposed to the compound test sample (FIG. 5f). This serves to demonstrate that the TCR A is fimctional and that lack of binding to sensor cell 1 is caused by the compound E. Similarly, it is demonstrated that the MHClpeptide complex B in sensor cell 2 is inaccessible for binding by passing the TCR specific for this complex (TCR G) over the flowcells (FIG. 5i). In the method described here which uses four flowcells, there is no control to ensure that this TCR is still functional. However, this control can be performed in a separate experiment or included in a fifth flowcell if provided.

Binding of TCR D (specific for MHC/peptide complex B) in sensor cell 3 demonstrates that compound E has not bound here and is specific for the MHC molecule of A and F (FIG. 5I).

After identification of compounds with the MHC specific binding described in FIG. 3, the HLA specificity could be farther verified by additional experiments, similar to that outlined in FIG. 5 but involving other MHC molecules and peptides.

The existing crystal structures of TCRs in complex with MHC/peptide have confirmed the generally-accepted view that TCRs must bind to both the presenting MHC molecule and the peptide antigen. The structural data shows that the main contacts to the MHC/peptide complex are made through the complementarity determining regions (CDRs) of the TCR. The CDR3, which is the most variable domain of the TCR, exclusively makes contact to the peptide. The CDR1 mainly makes contacts to the peptide, whereas the CDR2 mainly makes contacts to the MHC molecule (Garboczi, et al. Nature 384(6605):134-41 (1996); Garcia, et al. Science 274(5285):209-19 Issn: 0036-8075 (1996); Ding, et al. Immunity 8(4):403-11 (1998); Garboczi & Biddison Immunity 10(1):1-7 (1999)). The method of this embodiment of the present invention can allow the identification of compounds that inhibit or block the surface of a particular HLA molecule for binding by TCRs, irrespective of the peptide antigen specificity.

In many cases, the particular antigens involved in causing, for instance, autoimmune diseases, are not known. However, substantial information is available concerning the link between HLA type and disease. An impressive body of data has been accumulated which links specific HLA antigens with particular disease states (Table 1). The relationships are influenced by linkage disequilibrium, a state where closely linked genes on a chromosome tend to remain associated rather than undergo genetic randomisation in a given population, so that the frequency of a pair of alleles occurring together is greater than the product of the individual gene frequencies. This could result from natural selection favouring a particular haplotype or from insufficient time elapsing since the first appearance of closely located alleles to allow to become randomly distributed throughout the population.

With the odd exception, such as idiopathic hemochromatosis and congenital adrenal hyperplasia resulting from a 21-hydroxylase deficiency, HLA-linked diseases are intimately bound up with immunological processes. The HLA-D related disorders are largely autoimmune with a tendency for DR3 to be associated with organ-specific diseases involving cell surface receptors. A popular model of MHC and disease association is that efficient binding of autoantigens by disease-associated MHC molecules leads to a T cell-mediated immune response and the resultant autoimmune sequelae. Alternative models have also been put forward; for example, Ridgway and Fathman (Clin Immunol Immunopathol 86(1):3-10 (1998)) suggest that the association of MHC with autoimmunity results from “altered” thymic selection in which high-affinity self-reactive (potentially autoreactive) T cells escape negative selection.

TABLE 1
Association of HLA with disease

| Disease | HLA allele | Relative risk |
|---|---|---|
| (a) Class II associated | | |
| Hashimoto's disease | DR5 | 3.2 |
| Rheumatoid arthritis | DR4 | 5.8 |
| Dermatitis herpetiformis | DR3 | 56.4 |
| Chronic active hepatitis | DR3 | 13.9 |
| (autoimmune) | | |
| Coeliac disease | DR3 | 10.8 |
| Sjogren's syndrome | DR3 | 9.7 |
| Addison's disease (adrenal) | DR3 | 6.3 |
| Insulin-dependent diabetes | DR3 | 5.0 |
| | DR4 | 6.8 |
| | DR3/4 | 14.3 |
| | DR2 | 0.2 |
| Thyrotoxicosis (Grave's) | DR3 | 3.7 |
| Primary myxedema | DR3 | 5.7 |
| Goodpasture's syndrome | DR2 | 13.1 |
| Tuberculoid leprosy | DR2 | 8.1 |
| Multiple sclerosis | DR2 | 4.8 |
| (b) Class I, HLA-27 associated | | |
| Ankylosing spondylitis | B27 | 87.4 |
| Reiter's disease | B27 | 37.0 |
| Post-salmonella arthritis | B27 | 29.7 |
| Post-shigella arthritis | B27 | 20.7 |
| Post-yersinia arthritis | B27 | 17.6 |
| Post-gonococcal arthritis | B27 | 14.0 |
| Uveitis | B27 | 14.6 |
| Amyloidosis in rheumatoid | B27 | 8.2 |
| arthritis | | |
| (c) Other Class I associations | | |
| Subacute thyroiditis | Bw35 | 13.7 |
| Psoriasis vulgaris | Cw6 | 13.3 |
| Idiopathic hemochromatosis | A3 | 8.2 |
| Myasthenia gravis | B8 | 4.4 |

(Data from Ryder et al. HLA and disease Registry 1979. Tissue Antigens, supplement 1979)

Class II associations

A number of diseases have been linked to HLA Class II alleles, particularly DR2, DR3 and DR4. The most significant association appears to be that of dermatitis herpetiformis (coeliac disease of the skin), although associations have also been reported for coeliac disease itself, rheumatoid arthritis, insulin-dependent diabetes and multiple sclerosis. Other less common diseases with relatively high associations with HLA type are chronic active hepatitis, Sjogren's syndrome, Addison's disease and Goodpasture's syndrome.

The genetic contribution to the pathogenesis of rheumatoid arthritis

Rheumatoid arthritis is a chronic inflanmmatory disease that primarily affects the joints and surrounding tissues. Although the cause of rheumatoid arthritis is unknown, infectious, genetic, and endocrine factors may play a role. The disease can occur at any age, but the peak incidence of disease onset is between the ages of 25 and 55. Women are affected 3 times more often than men and incidence increases with age. Approximately 3% of the population is affected. The onset of the disease is usually slow, with fatigue, loss of appetite, weakness, and vague muscular symptoms. Eventually, joint pain appears, with warmth, swelling, tenderness, and stiffniess after inactivity of the joint. After having the disease for 10 to 15 years, about 20 percent of people will have had remission. Only 50% to 70% will remain capable of full-time employment and after 15 to 20 years, 10% of patients are invalids. The average life expectancy may be shortened by 3 to 7 years; factors contributing to death may be infection, gastrointestinal bleeding, and drug side effects. There is no known cure for rheumatoid arthritis and the disease usually requires life-long treatment. Current treatment includes various medications (including nonsteroidal anti-inflammatory drugs, gold compounds, immunosuppressive drugs), physical therapy, education, and possibly surgery aimed at relieving the signs and symptoms of the disease.

The association of HLA-DR4 or other HLA-DRB1 alleles encoding the shared (or rheumatoid) epitope has now been established in nearly every population. Similarly, the fact that the presence and gene dosage of HLA-DRBI alleles affect the course and outcome of rheumatoid arthritis has likewise been seen in most (although not all) studies. Susceptibility to develop rheumatoid arthritis maps to a highly conserved amino acid motif expressed in the third hypervariable region of different HLA-DRB 1 alleles. This motif, namely QKRAA, QRRAA or RRRAA helps the development of rheumatoid arthritis by an unknown mechanism. However, it has been established that the shared epitope can shape the T cell repertoire and interact with 70 kDa heat shock proteins (Reveille, Curr Opin Rheumatol 10(3):187-200 (1998)).

Coeliac disease and dermatitis herpetiformis

Coeliac disease is one of the most common gastrointestinal disorders, affecting between 1:90 to 1:600 persons in Europe. The disease is a permanent intolerance to ingested gluten that results in immunologically mediated inflammatory damage to the small-intestinal mucosa. Coeliac disease is associated with HLA and non-HLA genes and with other immune disorders, notably juvenile diabetes and thyroid disease. The classic sprue syndrome of steatorrhea and malnutrition coupled with multiple deficiency states may be less common than more subtle and often monosymptomatic presentations of the disease. Diverse problems such as dental anomalies, short stature, osteopenic bone disease, lactose intolerance, infertility, and nonspecific abdominal pain among many others may be the only manifestations of coeliac disease. The treatment of coeliac disease is lifelong avoidance of dietary gluten.

Recent studies using human genome screening in families with multiple siblings suffering from coeliac disease have suggested the presence of at least four different chromosomes in the predisposition to suffer from coeliac disease. Other studies based on cytokine gene polymorphisms have found a strong association with a particular haplotype in the TNF locus; this haplotype carries a gene for a high secretor phenotype of TNFα. In addition to the strong association of coeliac disease with HLA-DR3, there is also evidence for an association with HLA-DQ. Both HLA-DQ2 and HLA-DQ8 restricted gliadin-specific T cells have been shown to produce IFNγ, which appears to be an indispensable cytokine in the damage to enterocytes encountered in the small intestine, since the histological changes can be blocked by anti-IFNγ antibodies in vitro (Pena et al, Scand J Gastroenterol Suppl 225:56-8 (1998)).

Dermatitis herpetiformis (DH) is a pruritic, papulovesicular skin disease characterised in part by the presence of granular deposits of IgA at the dermal-epidermal junction, an associated gluten sensitive enteropathy, and a strong association with specific HLA types. Dermatitis herpetiformis is fairly uncommon, affecting around 1/10,000 persons in Europe and the US. Initial investigations revealed that 60% to 70% of patients with dermatitis herpetiformis expressed the HLA antigen B8 (normal subjects=21%). Further investigation of the HLA associations seen in patients with dermatitis herpetiformis has revealed an even higher frequency of the HLA class II antigens HLA-DR3 (OH=95%; normal=23%), HLA-DQw2 (DH=100%; normal=40%), and HLA-DPwI (DH=42%; normal=11%) (Hall and Otley, Semin Dermatol 10(3):240-5 (1991)). The association of the HLA-B8, HLA-DR3, HLA-DQw2 haplotype with Sjogren's syndrome, chronic hepatitis, CGrraves' disease, and other presumably immunologically mediated diseases, as welt as the evidence that some normal HLA-B8, HLA-DR3 individuals have an abnormal in vitro lymphocyte response to wheat protein and mitogens and have abnormal Fc-IgG receptor-mediated functions, suggests that this HLA haplotype or genes linked closely to it may confer a generalized state of immune susceptibility on its carrier, the exact phenotypic expression of which depends on other genetic or environmental determinants.

Genetic susceptibility factors in insulin-dependent diabetes mellitus

Diabetes mellitus is a disease of metabolic dysfunction, most notably dysregulation of glucose metabolism, accompanied by characteristic long-term vascular and neurolgical complications. Diabetes has several clinical forms, each of which has a distinct etiology, clinical presentation and course. Insulin-dependent diabetes mellitus (type I diabetes; IDDM) is a relatively rare disease (compared with non-insulin-dependent diabetes mellitus, NIDDM), affecting one in 250 individuals in the US where there are approximately 10,000 to 15,000 new cases reported each year. The highest prevalence of IDDM is found in northern Europe, where more than 1 in every 150 Finns develop IDDM by the age of 15. In contrast, IDDM is less common in black and Asian populations where the frequency is less than half that among the white population.

IDDM is characterised by absolute insulin deficiency, making patients dependent on exogenous insulin for survival. Prior to the acute clinical onset of IDDM with symptoms of hyperglycemia there is a long asymptomatic preclinical period, during which insulin-producing beta cells are progressively destroyed. The autoimmune destruction of beta cells is associated with lymphocytic infiltration. In addition, abnormalities in the presentation of MHC Class I antigens on the cell surface have been identified in both animal models and in human diabetes. This immune abnormality may explain why humans become intolerant of self-antigens although it is not clear why only beta cells are preferentially destroyed.

The genetics of IDDM is complex, but a number of genes have been identified that are associated with the development of IDDM. Some HLA loci (in particular DR3 and DR4) are associated with an increased risk of developing IDDM, whereas other loci appear to be protective. Substitution of alanine, valine or serine for the more usual aspartic acid residue at position 57 of the β-chain encoded by the HLA-DQ locus has also been found to be closely associated with the increased risk of developing IDDM, although different combinations of DQA1 and DQB1 genes confer disease risk to differing degrees (Zamani and Cassiman, Am J Med Genet 76(2):183-94 (1998)).

Genetics of multiple sclerosis

Multiple sclerosis (MS) is an inflammatory, demyelinating disease of the nervous system that is the most common cause of chronic neurological disability among young adults. MS is characterised by discrete demyelinating lesions throughout the CNS. The random nature of these lesions results in a wide variety of clinical features such as loss of sensations, muscle weakness, visual loss, cognitive impairment and fatigue. The mean age of onset is 30 years and females are more susceptible to MS than males by a factor that approaches 2:1. MS afflicts people almost worldwide, although there is epidemiologic variation in incidence and prevalence rates. The prevalence varies with latitude, affecting primarily northern Caucasian populations (e.g., 10 per 100,000 in southern USA, 300 per 100,000 in the Orkneys). Approximately 300,000 people are afflicted with MS in the U.S. and 400,000 in Europe.

In North European populations, MS has been linked with Class I HLA alleles A3 and B7 and with Class II HLA alleles DR2, DQw1, DQA1 and DQB1. Particular HLA alleles (especially DR2) are considered to be risk factors for MS, and not simply genetic markers for the population of origin. However, this relationship is not universal and MS is linked to alleles other than DR2 in some populations (e.g., Jordanian Arabs and Japanese). This suggests that there is some heterogeneity in the contribution of HLA polymorphisms to MS susceptibility. Although particular alleles increase the risk for MS, no specific allele has yet been identified that is necessary for the development of MS. Overall, the contribution of the MHC to MS risk is believed to be fairly minor (Ebers and Dyment, Semin Neurol 18(3):295-9 (1998)).

Class I associations

The best known association of Class I HLA types with disease is that of HLA-B27 with anklyosing spondylitis and the related group of spondylarthropathies. Of the other Class I associations, the most important is probably that of HLA-Cw6 with psoriasis, although associations have also been reported for subacute thyroiditis, idiopathic hemochromatosis and myasthenia gravis.

HLA-B27 and the seronegative spondylarthropathies

The seronegative spondylarthropathies include ankylosing spondylitis, Reiter's syndrome and reactive arthritis, psoriatic arthritis, arthritis associated with ulcerative colitis and Crohn's disease, plus other forms which do not meet the criteria for definite categories and are called undifferentiated. Seronegative spondylarthropathies have common clinical and radiologic manifestations:inflammatory spinal pain, sacroiliitis, chest wall pain, peripheral arthritis, peripheral enthesitis, dactylitis, lesions of the lung apices, conjunctivitis, uveitis and aortic incompetence together with conduction disturbances.

In the 25 years since the initial reports of the association of HLA-B27 with ankylosing spondylitis and subsequently with Reiter's syndrome/reactive arthritis, psoriatic spondylitis, and the spondylitis of inflammatory bowel disease, the association of HLA-B27 with the seronegative spondyloarthropathies has remained one of the best examples of a disease association with a hereditary marker. The association of HLA-27 with in ankylosing spondylitis is quite remarkable, where up to 95% of patients are of B27 phenotype as compared to around 5% in controls. The prevalence of spondylarthropathies is directly correlated with the prevalence of the HLA-B27 antigen in the population. The highest prevalence of ankylosing spondylitis (4.5%) has been found in Canadian Haida Indians, where 50% of the population is B27 positive. Among Europeans, the frequency of the B27 antigen in the general population ranges from 3 to 13% and the prevalence of ankylosing spondylitis is estimated to be 0.1-0.23% (Olivieri etal. Eur J Radiol27 Suppl 1:S3-6 (1998)).

Experimental evidence from humans and transgenic rodents suggests that HLA-B27 itself may be involved in the pathogenesis of the spondyloarthropathies, and population and peptide-specfficity analysis of HLA-B27 suggest it has a pathogenic finction related to antigen presentation. In Reiter's syndrome (reactive arthritis) and ankylosing spondylitis putative roles for infectious agents have been proposed. However, the mechanism by which HLA-B27 and bacteria interact to cause arthritis is not clear and there are no clear correlations between peptide sequence, differential binding to B27 subtypes and recognition by peptide-specific T cell receptors (Lopez-Larrea et al. Mol Med Today 4(12):540-9 (1998)).

HLA-B27 and uveitis

Uveitis involves inflammation of the uveal tract which includes the iris, ciliary body, and the choroid of the eye. Causes of uveitis can include allergy, infection, chemical exposure, trauma, or the cause may be unknown. The most common form of uveitis is anterior uveitis which affects the iris. The inflammation is associated with autoimmune diseases such as rheumatoid arthritis or ankylosing spondylitis. The disorder may affect only one eye and is most common in young and middle-aged people. Posterior uveitis affects the back portion of the uveal tract and may involve the choroid cell layer or the retinal cell layer or both. Inflammation causes spotty areas of scarring that correspond to areas with vision loss. The degree of vision loss depends on the amount and location of scarring.

In a recent study, Tay-Keamey et al (Am J Ophthalmol 121(1):47-56 (1996)) reviewed the records of 148 patients with HLA-B27-associated uveitis. There were 127 (86%) white and 21 (14%) nonwhite patients, and a male-to-female ratio of 1.5:1. Acute anterior uveitis was noted in 129 patients (87%), and nonacute inflammation was noted in 19 (13%). An HLA-B27-associated systemic disorder was present in 83 patients (58%), 30 of whom were women, and it was diagnosed in 43 of the 83 patients as a result of the ophthalmologic consultation. Thirty-four (30%) of 112 patients had a family history of a spondyloarthropathy.

The genetics of psoriasis

Psoriasis is a disease characterised by uncontrolled proliferation of keratinocytes and recruitment of T cells into the skin. The disease affects approximately 1-2% of the Caucasian population and can occur in association with other inflammatory diseases such as Crohn's disease and in association with human immunodeficiency virus infection. Non-pustular psoriasis consists of two disease subtypes, type I and type II, which demonstrate distinct characteristics. Firstly the disease presents in different decades of life, in type I before the age of 40 years and later in type II. Secondly, contrasting frequencies of HLA alleles are found:type I patients express predominantly HLA-Cw6, HLA-B57 and HLA-DR7, whereas in type II patients HLA-Cw2 is over-represented. Finally, familial inheritance is found in type I but not in type II psoriasis. The study of concomitant diseases in psoriasis contributes to deciphering the distinct patterns of the disease. Defence against invading microorganisms seems better developed in psoriatics than in controls. This evolutionary benefit may have caused the overall high incidence of psoriasis of 2% (Henseler. Arch Dermatol Res 290(9):463-76 (1998)).

Despite the HLA component, psoriasis in some families is inherited as an autosomal dominant trait with high penetrance. Susceptibility loci on other chromosomes have been identified following genoriie-wide linkage scans of large, multiply affected families although the extent of genetic heterogeneity and the role of environmental triggers and modifier genes is still not clear. The precise role of HLA also still needs to be defined. The isolation of novel susceptibility genes will provide insights into the precise biochemical pathways that control this disease. Such pathways will also reveal additional candidate genes that can be tested for molecular alterations resulting in disease susceptibility.

Thus, it can be seen that the association between certain HLA types and particular diseases has been well established. The best known of these is the association between the Class I molecule HLA-B27 and the spondylarthropathies, in particular ankylosing spondylitis. Despite the gene frequency of HLA-B27 being relatively high in Caucasians (3-13%), this group of diseases is not common and the overall significance of the association is therefore somewhat reduced. Similarly, the HLA-DR3 allele (present in approximately 11% %of the Caucasian population) is associated with a high risk (56.4) for the development of dermatitis herpetiformis, a relatively rare (1/10,000) skin disorder. However, there are associations between HLA types and more prevalent diseases with greater socioeconomic impact. For example, the relative risk of an individual with an HLA-DR4 allele developing rheumatoid arthritis is 5.8. Although this association is less than that between HLA-B27 and ankylosing spondylitis, rheumatoid arthritis affects approximately 3,% of the population and the HLA-DR4 allele has a gene frequency of nearly 17% in Caucasian Americans. Similarly, although coelic disease has a relatively low risk associated with the presence of HLA-DR3 (10.8), this is a comnon haplotype and coelic disease is a prevalent gastrointestinal disorder.

In summary, there are a number of clinical diseases where there is an association with a particular HLA type (or types). The diseases with the most significant association with HLA type tend to be somewhat uncommon. However, there are a number of examples where the prevalence of the disease combined with the frequency of the HLA allele in the population make the association more significant, even if the risk associated with the particular HLA type is relatively low.

Compounds that interfere with TCR binding to a particular HLA type molecule therefore have potential as immune inhibitors for the treatment of autoimmune diseases or the prevention of graft rejection, even in many situations where the causative antigen is not kmown.

Six class I and six class II HLA alleles are expressed in each human being. A panel of inhibitors, preventing TCR recognition and specific for various HLA type molecules, would furthermore enable a selective inhibition of parts of the immune responses in the body in situations where neither the causative peptide antigen or the HLA type involved are known. This could be used in studies to identify the HLA type involved in diseases, for which this information is not available. The inhibitors could also be tested for therapeutic effects in such cases, sequentially trying to inhibit a patient's HLA type-specific responses until a beneficial therapeutic effect was achieved.

C. Identification of compounds with the ability to block or inhibit HLA molecules for interactions with CD8 and CD4

FIG. 6 outlines a strategy for using SPR detection of CD8/CD4-MHC interactions to test, or screen for, compounds that inhibit or block the surface of a particular HLA molecule for coreceptor binding. Both CD8 and CD4 coreceptor binding are independent of the peptide antigens that are presented. The method has similar steps to the method described with reference to FIG. 1, the particular molecules being as follows:

Test ligand A=Class I HLA (including specific peptide antigen) for which a compound with binding specificity is sought.

Test ligand B=Class II HLA (including specific peptide antigen).

Test receptor C=CD8 receptor which recognises test ligand A

Test receptor D=CD4 receptor which recognises control ligand B

Test compound E=test compound having binding specificity for test ligand A

Test compound F=test compound having binding specificity for test ligand B.

Two MHC/peptide complexes, one belonging to the class I HLA type of molecules and on belonging to the class II HLA type of molecules, are produced as soluble protein complexes as described above. Soluble CD8 can be produced as described in Gao et al., Prot. Sci. 7:1245-49 (1998) and soluble CD4 multimers can be produced as described in the following Examples C3-C12, or as described in Allaway, et al. AIDS Res Hum Retroviruses 11(5):533-9 (1995) or Traunecker, et al Nature 339:68-70 (1989)).

Referring to FIG. 6a, the class I HLA complex A is immobilised in sensor cells 1 and 3, the class II HLA complex B in sensor cells 2 and 4 (FIG. 6B). When buffer is caused to flow over the sensor cells, the SPR readout is ‘flat’, indicating no changes in mass on any of the sensor surfaces.

In order to ensure that both the immobilised MHC/peptide complexes and the soluble coreceptors, i.e. CD8 (C) for the class I complex (A) immobilised in sensor cells 1 and 3 and CD4 (D) for the class HI complex (B) immobilised in sensor cells 2 and 4, are active, samples of the coreceptors (C and D) are passed over the sensor cell surfaces (FIGS. 6b and 6c). It is thus ensured that a signal is observed in the appropriate sensor cells in response to the relevant coreceptors:CD8 produces a signal in sensor cells 1 and 3, but not in cells 2 and 4, and CD4 produces a signal in sensor cells 2 and 4, but not in cells 1 and 3. These signal readouts serve to demonstrate that the soluble proteins employed are functional and specific.

Referring now to FIG. 6e, the compound sample to be tested is a mixture of different compounds; this could, for example, be a sample from a compound library. Alternatively, individual compounds could be passed sequentially over the sensor cells. The compound sample is are passed over sensor cells 1 and 2, but not over sensor cells 3 and 4 (FIG. 6d), which are retained for subsequent control purposes (FIGS. 6e and f).

If the test sample, as presumed in this example, contains two compounds that binds with high stability to each their HLA molecules, one E to the class I molecule A in sensor cell 1 and the other F to the class II molecule A in sensor cell 2. Higher constitutive levels of signal may be observed if the compound is of sufficient size for a change in mass to be detected. In the illustrated example, however, it is assumed that the compounds binding to the HLA molecules are too small to produce a detectable signal (FIG. 6d). As in the previous examples, it is without not important whether the compounds themselves produce a sufficient change in mass for detection or not, since the presence and specificity of the HLA molecule-compound interaction is demonstrated by subsequent testing with the relevant coreceptors (FIGS. 6e and f). With the compound E bound to the class I HLA/peptide complex A in sensor cell 1, CD8 can not bind here but can still bind in sensor cell 3, which was not exposed to the compound test sample (FIG. 6e). This serves to demonstrate that the CD8 is functional and that lack of binding to sensor cell 1 is caused by the compound E in the test sample. The same considerations apply to the class II HLA molecule/peptide B in sensor cell 2, with soluble CD4 specific for this complex being passed over the flowcells (FIG. 6f).

Thus, it is possible to screen for inhibitors of CD8 and CD4 binding to their respective HLA type molecules in the same experiment. Alternatively, only CD8 or CD4 screening could be performed and the extra flowcells used for other control ligands.

After identification of compounds with the HLA specific binding described in FIG. 6, the HLA specificity could be further verified by additional experiments, similar to those outlined in FIG. 6 but involving other HLA molecules and peptides.

Because of the fast off-rates of CD8-class I HLA/peptide interactions (Wyer et al. Immunity 10:219-225 (1999)), and the presumed fast off-rates of CD4-class II HLA/peptide interactions (Davis, et al. Imm. Rev. 163:217-36 (1998)), the binding events are detected only while, and immediately after, the samples of the soluble coreceptors C and D are flowed over the sensor surfaces. Almost immediately after the soluble coreceptor samples have passed through the sensor cells, are the MHC/peptide complexes A and B are left free to be bound by another compound (see FIGS. 6b and 6c).

The vast majority of class I-restricted T cell responses require signalling by CD8 which is activated through its binding to HLA (Zamoyska, et al. Nature 342(6247): 278-81 (1989); Sewell et al. Nature Medicine 5:399-404 (1999)). Similarly, the vast majority of class II-restricted T cell responses require signalling by CD4. Therefore, compounds that interfere with either CD8-class I HLA interactions or with CD4-class II HLA interactions can be used as immune inhibitors for the respective branches of the cellular immune system. If inhibition of both branches of the cellular immune system is required, the two types of compounds could be used together. These types of immune inhibition, administered alone or together with other types of immune inhibitors, could potentially offer substantial advantages over current immune inhibition therapeutics like, for example, steroids. The compounds will exercise their immune inhibitory effects through their specificities for class I and class II HLA type molecules, respectively, and therefore should be less likely to cause the unwanted side-effects associated with conventional therapeutics.

EXAMPLE C1

The use of BIAcore biomolecular interaction analysis for screening for compounds which inhibit the interaction between CD8 and HLA-A2

CD8 is a membrane bound T cell co-receptor molecule, which, along with the T cell receptor, binds to class I MHC molecules (eg. HLA-A2) to initiate T cell activation. In the present example, a recombinant soluble form of CD8 was used (sCD8αα), the preparation of which is described in WO99/21576. HLA-A2 was prepared as described in Example A1. The interaction between MHC molecule HLA-A2 and sCD8αα shows extremely rapid kinetics (Wyer et al. (1999) Immunity 10:219-225) which prevents the use of conventional screening strategies.

The BIAcore 3000 ™ system was used to screen a small compound library containing 96 compounds for compounds which inhibit the interaction between sCD8αα and HLA-A2.

sCD8αα was transferred into HBSE buffer (10 mM HEPES pH 7.4, 150 mM NaCl, 3 mM EDTA) using gel filtration chromatography (Phanmacia 10/30 Superdex 200 HR column) and concentrated to ˜5 mg/ml using a Millipore ultrafree centrifugal concentrator.

A CM-5 sensor chip was docked onto the BIAcore 3000™. Streptavidin was coupled to the carboxymethyl surface using standard amine coupling. The chip surface was activated with 0.2M EDAC/0.05M NHS, followed by binding of streptavidin (0.25 mg/ml in 10 mM sodium acetate pH 5.0) and saturation of unoccupied site with 1 M ethylenediamine.

HLA-A2 (prepared as described in Example Al and tagged with a biotin molecule) was immobilised on the streptavidin-coated surface until a response of approximately 5000 RU was observed.

Mixtures of compounds were solubilised in DMSO to a concentration of 0.5 mg/ml, and then diluted into BIAcore buffer to a concentration of 50 μg/ml of each compound in the mixture, including a total of 8% DMSO. This was then diluted 10× in BIAcore buffer to make a final working solution (5 μg/ml per compound, 0.8% DMSO).

BIAcore 3000™ was run at a flow rate of 10 μl/min using BIAcore buffer. Compounds, DMSO solutions, or sCD8αα solutions were injected using the BIAcore COINJECT program. This enables sCD8αα to be injected directly following the injection of compound with no BIAcore buffer flowing over the sensor surface in between. Data were recorded automatically and were analysed using BIAevaluation software. sCD8αα specific responses were calculated, taking account of any variance in the baseline between the two cells. The loss of signal was calculated as a percentage of the initial sCD8αα specific response.

FIG. 7 shows the BIAcore trace of the trial screen of 96 compounds, for the sCD8αα —HLA-A2 interaction, using the COINJECT program on the BIAcore 3000™. No inhibition of sCD8αα binding was observed after any of the compound injections. An overall signal loss of 3.6% occurred during this trial over the course of 12 injections of compound mixtures (total of 96 compounds).

### EXAMPLE C2

The use ofBIAcore molecular interaction analysis for screening compounds to inhibit the sCD8αα—HLA-A2 interaction.

Reagents and CM-5 sensor chips were prepared as described in Example C1. The BIAcore robot was used for screening a library of 10000 compounds. Compounds were purchased from Cambridge Drug Discovery and plated out in 96 well micro litre plates in mixtures of 5 compounds. Compounds were prepared as described in Example C1, except that BIAcore running buffer +1.25% DMSO (v/v) was used as a diluent.

A BIAcore screening macro was written using TRANSFER, MIX and QUICKINJECT commands. A compound mixture (30 μl) was transferred to a well containing sCD8αα (10 μl), and mixed. An aliquot of the mixture (15 μl) was injected over a test flow cell and control flow cell (presence and absence of bound HLA-A2 respectively) at a flow rate of 30 μl/min using the BIAcore QUICKINJECT program.

Data were recorded automatically and were analysed using the BIAevaluation software. sCD8αα specific responses were calculated, taking into account differences between the control and test flow cell.

FIGS. 8a and 8b show results from two plates containing 440 compounds in mixtures of five per well in a screen of 10000 compounds. There was a small loss of signal over each of the runs, but this did not interfere with the ability to distinguish potential hits amongst the data. FIG. 8a illustrates the ability of the screening methodology to generate reproducible results over a series of 440 compounds. Each point (♦) is the relative increase in response of the BIAcore to sCD8αα, in the presence of potential inhibitors. The lines indicate ±15% from a trendline drawn through the data points. None of this batch of 440 compounds significantly effects the interaction between sCD8αα and HLA-A2.

FIG. 8b shows that most of the mixtures of compounds do not affect the interaction between HLA-A2 and sCD8αα (♦). However, four compound mixtures promote the interaction (□), and two decrease the interaction (◯).

The production of a multimeric CD4 complex is described in the following non-limiting examples C3-12. The materials and methods used in these examples are as follows:

Restriction enzymes (ApaI, EcoRI, NdeI, and Xmal) were from New England Biolabs. All restrictions were done in 20 μl Tris-Acetate buffer (33 mM Tris-Acetate pH 7.9; 66 mM K-Acetate; 10 mM Mg-Acetate; 0,5 mM DDT; 100 μg autoclaved gelatin). DNA fragments were purified from TBE-agarose gels by electro transfer onto GF/C, eluted by centrifugation and purified by extraction with phenol:chloroform:isoarnylic alcohol (25:24:1) and spin column chromatography on sephadex G-50 columns equilibrated in TE-buffer (10 mM Tris-HCl pH 8.0; 1 mM, EDTA). Lyophilised oligo nucleotides were purchased from MIWG-Biotech and dissolved at 40 ,μM in H₂O. Oligos (except the ones generating the 5′ ends of the individual cassettes) were phosphorylated individually at 4 μM in 10 μl of T4 DNA Ligase Buffer (Boehringer Mannheim) supplemented with ATP to 1 mM and 0.5 units T4 polynucleotide kinase. The kinase was inactivated by heat denaturation 15 minutes at 94° C. Oligos were combined pairwise and annealed by slow cooling from 90° C. to room temperature. Oligo pairs making up individual domains were combined, supplemented with 1 volume of T4 DNA ligase buffer (Boehringer Mannheim) containing 1 mM ATP and 0.2 unit/μl of T4 DNA ligase (Boehringer Mannheim) and ligated for 5 hours with alternating temperatures (15° C. for 10 minutes/30° C. for 10 minutes). After ligation, the casettes were purified by extraction with phenol:chloroform:isoamylic alcohol (25:24:1) and precipitated by addition of 0.1 vol Na-acetate pH 5.2 and 2 vol absolute ethanol. The casettes were separated on 2% Mataphor agarose™ and fragments of the right size were purified as described above for restriction fragments. Ligations were dore using a Rapid T4 DNA Ligase kit(Boehringer Mannheim) according to the manufacturer's instruction. All constructions were transformed into E. coli XL-1 Blue™ (Stratagene) according to standard techniques. Plasmids were prepared from positive colonies grown in 20 ml LB medium (10 g Bacto Tryptone, 5 g Bacto Yeast Extract, and 10 g NaCl per litre) using Qiaprep Spin Miniprep Kit according to the instructions provided by the manufacturer. Automated sequencing reactions with ABI Prism Big Dye™ were done at the Sequencing Facility, Department of Biochemistry at Oxford University.

### EXAMPLE C3

Construction of plasmid encoding the Hinge-domain.

Gene casettes for the generation of fusion proteins were built from oligonucleotides and inserted into the E. coli expression plasmid pGMT7. This plasmid uses the T7 promoter to drive expression of recombinant proteins in Escherishia coli in response to the synthetic inducer IPTG. The oligonucleotide approach allows the use of codons preferred by E. coli, as well as incorporation of restriction sites wherever appropriate.

Initially, the Hinge domain plasmid was built by ligating the phosphorylated and annealed oligo pair HingeF and HingeB (see FIG. 11a) into the NdeI- and EcoRI-sites of pGMT7 resulting in plasmid pEX122. The DNA sequence of the hinge-coding region of the plasmid was verified by automated sequencing.

### EXAMPLE C4

Construction ofplasmid encoding the Hinge-dimerisation-domains.

The oligos of the dimerisation cassette (indicated by the alternating pattern of boxes in FIG. 11b) were assembled and ligated into the ApaI- and EcoRI sites of pEX122 described above. The sequence of the Hinge- and dimerisation domain coding regions of the resulting plasmid, pEX123, was verified by automated sequencing.

### EXAMPLE C5

Construction ofplasmid encoding the Hinge-trimerisation-domains.

The oligos of the trimerisation cassette (indicated by the alternating pattern of boxes in FIG. 11c) were assembled and ligated into the ApaI- and EcoRI sites of pEX122 described above. The sequence of the Hinge- and trimerisation domain coding regions of the resulting plasmid, pEX124, was verified by automated sequencing.

### EXAMPLE C6

Construction ofplasmid encoding the Hinge-tetramerisation-domains.

The oligos of the tetramerisation cassette (indicated by the alternating pattern of boxes in FIG. 11d) were assembled and ligated into the ApaI- and EcoRI sites of pEX122 described above. The sequence of the Hinge- and tetramerisation domain coding regions of the resulting plasmid, pEX125, was verified by automated sequencing.

### EXAMPLE C7

Construction ofplasmid encoding the Hinge-biotinylation-domains.

The oligos of the biotinylation cassette (shown in FIG. 11e) were annealed and ligated into the ApaI- and EcoRI sites of pEX122 described above. The sequence of the Hinge- and dimerisation biotinylation domain coding regions of the resulting plasmid, pEX126, was verified by automated sequencing.

### EXAMPLE C8

Construction of E. coli expression plasmid encoding the extracellular domains 1 and 2 of human CD4.

The gene encoding the extracellular domains 1 and 2 of human CD4 was amplified from a plasmid containing the complete human CD4 gene sequence. The primers used are shown in FIG. 12. A number of silent mutations (indicated by underlining in FIG. 12) were introduced in the 5′-end of the gene in order to facilitate expression initiation in E. coli. The PCR fragment was subdloned into pGMT7 between the NdeI-site and the HindIII-site. The sequence of the resulting expression plasmid, pEX121, was verified by sequencing.

### EXAMPLE C9

Construction ofplasmid encoding the CD4-dimer.

The CD4-gene fragment from pEX121 was amplified by PCR using the primers OX 332 and OX334 (see FIG. 12) and subcloned between the NdeI site and the Xmal site of pEX123. The sequence of the resulting expression plasmid, pEX133, was verified by sequencing.

### EXAMPLE C10

Construction ofplasmid encoding the CD4-trimer.

The CD4 coding fragment of pEX133 was excised by restriction with NdeI and Xmal and subcloned into pEX124 opened by restriction with the same enzymes. The sequence of the resulting expression plasmid, pEX134, was verified by sequencing.

### EXAMPLE C11

Construction ofplasmid encoding the CD4-tetramer.

The CD4 coding fragment of pEX133 was excised by restriction with NdeI and Xmal and subcloned into pEX125 opened by restriction with the same enzymes. The sequence of the resulting expression plasmid, pEX135, was verified by sequencing.

### EXAMPLE C12

Construction ofplasmid encoding biotinylation-tagged CD4.

The CD4 coding fragment of pEX133 was excised by restriction with NdeI and XmaI and subcloned into pEX126 opened by restriction with the same enzymes. The sequence of the resulting expression plasmid, pEX136, was verified by sequencing.

The prior art documents mentioned herein are incorporated to the fullest extent permitted by law. Preferred features of each aspect of the invention are as for each of the ether aspects mutatis mutandis.

1. A method of sequentially screening candidate compounds for compounds with the ability to inhibit a receptor-ligand interaction having fast binding kinetics, the method comprising the steps of:
a) optionally contacting the receptor with the ligand, the receptor being immobilised so that binding of the ligand therewith can be detected in an interfacial optical assay, detecting by interfacial optical assay the binding of the ligand to the receptor, and washing the ligand from the receptor;
b) contacting an n^{th} candidate compound with the immobilized receptor;
c) optionally washing the receptor at a predetermined stringency to remove the n^{th} candidate compound if it has too low an affinity for the receptor;
d) contacting the receptor, which may or may not have the n^{th} candidate compound bound to it, with the ligand, and detecting by interfacial optical assay whether or not the ligand or ligand-compound complex has bound to the receptor or receptor-compound complex; and
e) either i) if the ligand has bound, deducing that the n^{th} compound does not inhibit the receptor-ligand interaction, optionally washing the receptor, incrementing n, and returning to optional step a) or step b), or ii) if the ligand has not bound, deducing that the n^{th} compound inhibits the receptor-ligand interaction.

2. A method as claimed in claim 1, wherein the interfacial optical assay is surface plasmon resonance.

3. A method as claimed in claim 1 or claim 2, wherein step a) is not optional.

4. A method as claimed in any preceding claim, wherein step c) is not optional.

5. A method as claimed in any preceding claim, wherein the stringency of washing is predetermined according to the time taken for washing.

6. A method as claimed in any preceding claim wherein, in step b), the receptor is contacted with a sample comprising a predetermined plurality of candidate compounds.

7. A method as claimed in claim 6, further comprising, if the sample causes inhibition of receptor-ligand binding, returning to optional step a) or step b) for each candidate compound in the sample.

8. A method as claimed in any preceding claim, further comprising the steps of:
a1) optionally contacting a control receptor with a control ligand, the control receptor being immobilised so that binding of the control ligand therewith can be detected in an interfacial optical assay, detecting by interfacial optical assay the binding of the control ligand to the control receptor, and washing the control ligand from control the receptor;
b1) contacting the n^{th} candidate compound with the immobilised control receptor;
c1) optionally washing the control receptor at the predetermined stringency;
d1) contacting the control receptor with the control ligand, and detecting by interfacial optical assay whether or not the control ligand or control ligand-compound complex has bound to the control receptor or control receptor-compound complex.

9. A method as claimed in claim 8, wherein step b1) is carried out simultaneously with step b).

10. A method as claimed in claim 8 or claim 9, wherein step c1) is carried out simultaneously with step c).

11. A method as claimed in claim 8, 9 or 10, wherein steps a1) and d1) are carried out before or after steps a) and d) respectively.

12. A method as claimed in any preceding claim, wherein the receptor-ligand interaction is the interaction between MHGC/peptide complex and T cell receptor.

13. A method as claimed in any one of claims 1 to 11, wherein the receptor-ligand interaction is the interaction between MHCY peptide complex and CD8 coreceptor.

14. A method as claimed in any one of claims 1 to 11, wherein the receptor-ligand interaction is the interaction between MHClpeptide complex and CD4 coreceptor.

15. A method as claimed in claim 12, 13 or claim 14, wherein the MHC-peptide complex, T cell receptor, CD8 coreceptor or CD4 coreceptor is modified to allow increased avidity of binding, preferably without inducing changes in the affinity of the interaction.

16. A method as claimed in claim 15, wherein MHC-peptide complex, T cell receptor, CD8 coreceptor or CD4 coreceptor is provided as a multivalent complex comprising a plurality of monomeric MHC-peptide complex, T cell receptor, CD8 coreceptor or CD4 coreceptor molecules, respectively.

17. A method as claimed in claim 16, wherein the complex is a multimer, such as a di-, tri- or tetramer.

18. A method as claimed in claim 16 or claim 17, wherein the complex comprises a multimerisation module attached or associated with each monomer in the complex.

19. A method as claimed in claim 18, wherein the multimerisation module comprises a coiled coil domain.

20. A method as claimed in claim 18, wherein the multimerisation module comprises multivalent linker molecule such as avidin, streptavidin or extravidin.

21. A method as claimed in claim 20, wherein each MHC-peptide complex, T cell receptor, CD8 coreceptor or CD4 coreceptor monomer in the complex is derived from a fusion protein comprising an amino acid recognition sequence for a modifying enzyme, such as BirA.

22. A molecule selected from MHC, NMC-peptide complex, T cell receptor, CD8 and CD4 immobilised for use in an interfacial optical assay.
